(19) United States
(12) Patent Application Publication
(10) Pub. No.: US 2010/0152531 A1
(43) Pub. Date: Jun. 17, 2010

(54) IMPLANTABLE MEDICAL DEVICE FOR DRUG DELIVERY AND METHOD OF USE

(75) Inventors: Amy M. Goodman, San Francisco, CA (US); Peter M. Martin, Mountain View, CA (US); William Mavity, Los Altos, CA (US); Russell Ivanhoe, Palo Alto, CA (US); Matthew G. Fishler, Sunnyvale, CA (US); Craig Mar, Fremont, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: Paracor Medical, Inc., Sunnyvale, CA (US)

(21) Appl. No.: 12/634,974

(22) Filed: Dec. 10, 2009

Related U.S. Application Data
(60) Provisional application No. 61/121,800, filed on Dec. 11, 2008.
(60) Provisional application No. 61/181,531, filed on May 27, 2009.

(51) Int. Cl. A61M 31/00; A61F 2/00

(52) U.S. Cl. 600/37

(57) ABSTRACT

An apparatus and method of preparing a cardiac harness for use in controllably delivering a medicament such as an mTOR inhibitor or aldosterone blockade from the surface of the cardiac harness to the epicardium of a patient's heart for the site-specific treatment of cardiac and non-cardiac maladies. The delivery of the medicament from the cardiac harness is achieved by: coating the medicament on the surface of the cardiac harness; coating the cardiac harness with a polymer material or dielectric material and impregnating the coating with the medicament; or by loading the medicament into an implant attached to the cardiac harness.

### CROSS-REFERENCES TO RELATED APPLICATIONS

This application claims priority from U.S. Provisional Application No. 61/121,800, filed Dec. 11, 2008 and U.S. Provisional Application No. 61/181,531, filed May 27, 2009, each incorporated by reference in its entirety.

### BACKGROUND OF THE INVENTION

The present invention relates to a device for treating heart failure. More specifically, the invention relates to a cardiac harness that is configured to be fit around at least a portion of a patient's heart and is associated with electrodes attached to a power source for use in defibrillation or pacing. The cardiac harness is further combined with medically beneficial medicaments and a system for delivery of the medicaments so that the beneficial medicaments are controllably released to a patient's heart over an appropriate time horizon. Such a combination will serve to augment and/or extend the efficacy of the cardiac harness and the medicaments used.

Congestive heart failure (“CHF”) is characterized by the failure of the heart to pump blood at sufficient flow rates to meet the metabolic demand of tissues, especially the demand for oxygen. One characteristic of CHF is remodeling of at least portions of a patient's heart. Remodeling involves physical change to the size, shape and thickness of the heart wall. For example, a damaged left ventricle may have some localized thinning and stretching of a portion of the myocardium. The thinned portion of the myocardium often is functionally impaired, and other portions of the myocardium attempt to compensate. As a result, the other portions of the myocardium may expand so that the stroke volume of the ventricle is maintained notwithstanding the impaired zone of the myocardium. Such expansion may cause the left ventricle to assume a somewhat spherical shape.

Cardiac remodeling often subjects the heart wall to increased wall tension or stress, which further impairs the heart's functional performance. Often, the heart wall will dilate further in order to compensate for the impairment caused by such increased stress. Thus, a cycle can result, in which dilation leads to further dilation and greater functional impairment.

Historically, congestive heart failure has been managed with a variety of drugs. Devices have also been used to improve cardiac output. For example, left ventricular assist pumps help the heart to pump blood. Multi-chamber pacing has also been employed to optimally synchronize the beating of the heart chambers to improve cardiac output. Various skeletal muscles, such as the latissimus dorsi, have been used to assist ventricular pumping. Researchers and cardiac surgeons have also experimented with prosthetic “girdles” disposed around the heart. One such design is a prosthetic “sock” or “jacket” that is wrapped around the heart.

Patients suffering from congestive heart failure often are at risk to additional cardiac failures, including cardiac arrhythmias. When such arrhythmias occur, the heart must be shocked to return it to a normal cycle, typically by using a defibrillator. Implantable cardioverter/defibrillators (ICD's) are well known in the art and typically have a lead from the ICD connected to an electrode implanted in the right ventricle. Such electrodes are capable of delivering a defibrillating electrical shock from the ICD to the heart.

Other prior art devices have placed the electrodes on the epicardium at various locations, including on or near the epicardial surface of the right and left heart. These devices also are capable of distributing an electrical current from an implantable cardioverter/defibrillator for purposes of treating ventricular defibrillation or hemodynamically stable or unstable ventricular tachyarrhythmias.

Patients suffering from congestive heart failure may also suffer from cardiac failures, including bradycardia and tachycardia. Such disorders typically are treated by both pacemakers and implantable cardioverter/defibrillators. The pacemaker is a device that paces the heart with timed pacing pulses for use in the treatment of bradycardia, where the ventricular rate is too slow, or to treat cardiac rhythms that are too fast, i.e., anti-tachycardia pacing. As used herein, the term “pacemaker” is any cardiac rhythm management device with a pacing functionality, regardless of any other functions it may perform such as the delivery cardioversion or defibrillation shocks to terminate atrial or ventricular fibrillation. Particular forms and uses for pacing/sensing can be found in U.S. Pat. No. 6,574,506 (Kramer et al.) and U.S. Pat. No. 6,223,079 (Bakels et al.); and U.S. Publication No. 2003/0130702 (Kramer et al.) and U.S. Publication No. 2003/0195575 (Kramer et al.), the entire contents of which are incorporated herein by reference thereto.

In addition, particular forms and uses for cardiac harnesses used for treating CHF and for defibrillating and/or pacing/sensing can be found in U.S. Pat. No. 7,155,295 (Lilip Lau et al.), the entire contents of which is incorporated herein by reference thereto.

In addition to the benefits derived from the cardiac harness disclosed herein, including the electrically active harness (i.e., defibrillation, pacing, sensing), the harness can be used to deliver drugs to the surface of the heart. Many drugs used for heart failure and other cardiac and non-cardiac maladies may have complications and side effects when delivered systemically. Most drugs do not spread out evenly through the body. The drugs may have limited onset and breakdown times when delivered systemically. A variety of body-wide factors may affect the effectiveness of the dose when delivered systemically, including but not limited to, time required to enter blood stream, amount entering bloodstream, and time to leave the bloodstream or be metabolized.

Delivery of appropriate and beneficial medicaments directly to the surface of the heart may allow lower overall doses to be utilized, as the delivery is directly to or near the site of intended impact. As used herein, the term “beneficial medicament” is an agent that assists in the treatment, cure, relief or prevention of disease or disorders of the heart or surrounding tissue. Beneficial medicaments may include one or more therapeutic agents, cellular material, and/or combinations thereof. Any therapeutic compound that affects the heart, coronary vessels, or surrounding tissue may be suitable to use in combination with the cardiac harness.

The present invention solves the problems associated with prior art devices relating to the delivery of beneficial medicaments, such as drugs, from implantable medical devices for the site-specific treatment of cardiac and non-cardiac maladies.

### SUMMARY OF THE INVENTION

In accordance with the present invention, a cardiac harness is combined with beneficial medicaments and a system for delivery of the medicaments so that the beneficial medicaments are controllably released to a patient's heart over an appropriate time horizon. Such a combination will serve to augment and extend the efficacy of the cardiac harness and the medicaments used.

In one embodiment, a cardiac harness is combined with an mTOR inhibitor delivered locally to one or more specific target areas on or around the heart. In this embodiment, the mTOR inhibitor is delivered directly to the epicardial surface of the heart by the cardiac harness. In another embodiment, a cardiac harness is combined with aldosterone blockade delivered locally to one or more specific target areas on or around the heart. In this embodiment, the aldosterone blockade is used in conjunction with standard care therapies which include the use of ACE inhibitors and β-blockers. The aldosterone blockade has a dose range of 0.1 to 200 mg per day targeted, and the drugs are delivered directly to the epicardial surface of the heart by the cardiac harness. In one embodiment, the pericardium remains intact over the cardiac harness, thereby helping to keep the drugs in contact with the epicardium.

In another embodiment, a polymer material such as, for example, a silicon rubber layer, coats the cardiac harness. The polymer material is then coated with an mTOR inhibitor coating or a non-biodegradable aldosterone blockade coating.

In another embodiment, a polymer material on the cardiac harness is itself impregnated with a beneficial medicament such as an mTOR inhibitor. In this embodiment, over time the mTOR inhibitor elutes directly from the impregnated polymer coating. Alternately, a dielectric coating on the cardiac harness is impregnated with a beneficial medicament such as aldosterone blockade. In this embodiment, the aldosterone blockade elutes directly from the impregnated dielectric coating over time.

In yet another embodiment, a free-standing biodegradable plug or implant is attached to the cardiac harness. In this embodiment, over time the biodegradable beneficial medicament elutes, degrades, and separates itself from the plug or implant.

In another embodiment, the cardiac harness has longitudinal lumens through which the mTOR inhibitor or aldosterone blockade is injected directly onto the epicardial surface of the heart.

### BRIEF DESCRIPTION OF THE DRAWINGS

FIG. 1 depicts a schematic view of a heart with a prior art cardiac harness placed thereon.

FIGS. 2A-2B depict a spring hinge of a prior art cardiac harness in a relaxed position and under tension.

FIG. 3 depicts a prior art cardiac harness that has been cut out of a flat sheet of material.

FIG. 4 depicts the prior art cardiac harness of FIG. 3 formed into a shape configured to fit about a heart.

FIG. 5A depicts a flattened view of one embodiment of the cardiac harness showing two panels connected to two electrodes.

FIG. 5B depicts a cross-sectional view of an electrode.

FIG. 5C depicts a cross-sectional view of an electrode.

FIG. 5D depicts a cross-sectional view of an electrode.

FIG. 6A depicts a cross-sectional view of an undulating strand or ring.

FIG. 6B depicts a cross-sectional view of an undulating strand or ring.

FIG. 6C depicts a cross-sectional view of an undulating strand or ring.

FIG. 7A depicts an enlarged plan view of a cardiac harness showing three electrodes separating three panels, with the far side panel not shown for clarity.

FIG. 7B depicts an enlarged partial plan view of the cardiac harness of FIG. 7A showing an electrode partially covered with a dielectric material which also serves to attach the panels to the electrode.

FIG. 8A depicts a transverse cross-sectional view of the heart showing the position of electrodes for defibrillation and/or pacing/sensing functions.

FIG. 8B depicts a transverse cross-sectional view of the heart showing the position of electrodes for defibrillation and/or pacing/sensing functions.

FIG. 8C depicts a transverse cross-sectional view of the heart showing the position of electrodes for defibrillation and/or pacing/sensing functions.

FIG. 8D depicts a transverse cross-sectional view of the heart showing the position of electrodes for defibrillation and/or pacing/sensing functions.

FIG. 9 depicts a plan view of one embodiment of a cardiac harness having panels separated by and attached to flexible coils.

FIG. 10 depicts a flattened plan view of a cardiac harness similar to that of FIG. 9 but with fewer panels and coils.

FIG. 11 depicts a plan view of one embodiment of a cardiac harness having panels separated by and attached to flexible coils.

FIG. 12 depicts a plan view of a cardiac harness similar to that shown in FIG. 11 mounted on the epicardial surface of the heart.

FIG. 13 depicts a perspective view of a cardiac harness similar to that of FIG. 9 where the harness has been folded to reduce its profile for minimally invasive delivery.

FIG. 14 depicts the cardiac harness of FIG. 13 in a partially bent and folded condition to reduce its profile for minimally invasive delivery.

FIG. 15A depicts an enlarged plan view of a cardiac harness showing continuous undulating strands with electrodes overlaying the strands.

FIG. 15B depicts an enlarged partial plan view of the cardiac harness of FIG. 15A showing continuous undulating strands with an electrode overlying the strands.

FIG. 15C depicts a partial cross-sectional view taken along lines 15C-15C showing the electrode and undulating strands.

FIG. 15D depicts a partial cross-sectional view taken along lines 15D-15D showing the undulating strands in notches in the electrode.

FIG. 16 depicts a top view of a fixture for winding wire to construct the cardiac harness.

FIG. 17 depicts a plan view of a portion of a cardiac harness showing panels separated by electrodes.

FIGS. 18A, 18B and 18C depict various views of a mold used for injecting a dielectric material around the cardiac harness and the electrodes.

FIGS. 19A, 19B and 19C depict various views of molds used in injecting a dielectric material around the cardiac harness and the electrodes.

FIG. 20 depicts a top view of a portion of an electrode having a metallic coil winding.

FIG. 21 depicts a side view of the electrode portion shown in FIG. 20.

FIG. 22 depicts a cross-sectional view taken along lines 22-22 showing lumens extending through the electrode.

FIG. 23 depicts a cross-sectional view taken along lines 23-23 depicting another embodiment of lumens extending through the electrode.

FIG. 24 depicts a top view of a portion of an electrode having multiple coil windings.

FIG. 25A depicts a side view of a portion of a defibrillator electrode combined with a pacing/sensing electrode.

FIG. 25B depicts a top view of the electrode portion of FIG. 25A.

FIGS. 26A-26C depict various views of a defibrillator electrode combined with a pacing/sensing electrode.

FIG. 27 depicts a side view of an introducer for delivering the cardiac harness through minimally invasive procedures.

FIG. 28 depicts a perspective end view of a dilator with the cardiac harness releasably positioned therein.

FIG. 29 depicts an end view of the introducer with the cardiac harness releasably positioned therein.

FIG. 30 depicts a schematic cross-sectional view of a human thorax with the cardiac harness system being delivered by a delivery device inserted through an intercostal space and contacting the heart.

FIG. 31 depicts a plan view of the heart with a suction device releasably attached to the apex of the heart.

FIG. 32 depicts a plan view of the heart with the suction device attached to the apex and the introducer positioned to deliver the cardiac harness over the heart.

FIG. 33 depicts a plan view of the cardiac harness being deployed from the introducer onto the epicardial surface of the heart.

FIG. 34 depicts a plan view of the heart with the cardiac harness being deployed from the introducer onto the epicardial surface of the heart.

FIG. 35 depicts a plan view of the heart with the cardiac harness having electrodes attached thereto, surrounding a portion of the heart.

FIG. 36 depicts a schematic view of the cardiac harness assembly mounted on the human heart together with leads and an ICD for use in defibrillation or pacing.

FIG. 37 depicts an exploded a side view of a delivery system with the introducer tube, dilator tube, and ejection tube shown prior to assembly.

FIG. 38 depicts a cross-sectional view of the introducer tube taken along lines 38-38.

FIG. 39 depicts a cross-sectional view taken along lines 39-39 showing the cross-section of the dilator tube.

FIG. 40 depicts a cross-sectional view taken along lines 40-40 extending through the plate of the ejection tube and showing the various lumens in the plate.

FIG. 41 depicts a cross-sectional view taken along lines 41-41 of the proximal end of the ejection tube.

FIG. 42 depicts a longitudinal cross-sectional view and schematic of the ejection tube with the leads from the electrodes extending through the lumens in the plate and the tubing from the suction cup extending through a lumen in the plate.

FIG. 43 depicts a plan view of a portion of a cardiac harness showing panels separated by electrodes and implants loaded into the cells of the cardiac harness.

FIGS. 44-54 depict exemplary cross-sectional views of various constructions of cardiac harnesses adapted to deliver a medicament to the heart.

### DETAILED DESCRIPTION OF THE PREFERRED EMBODIMENTS

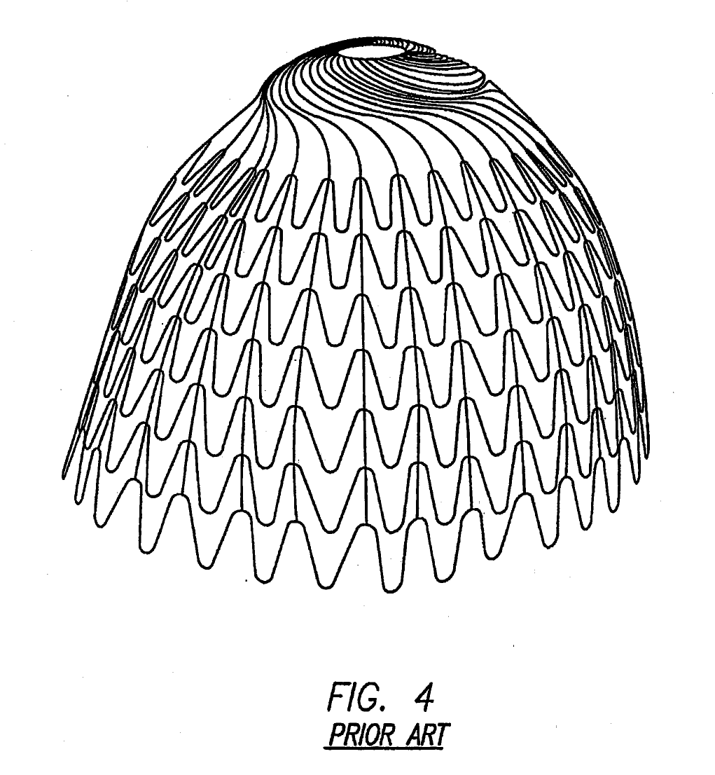

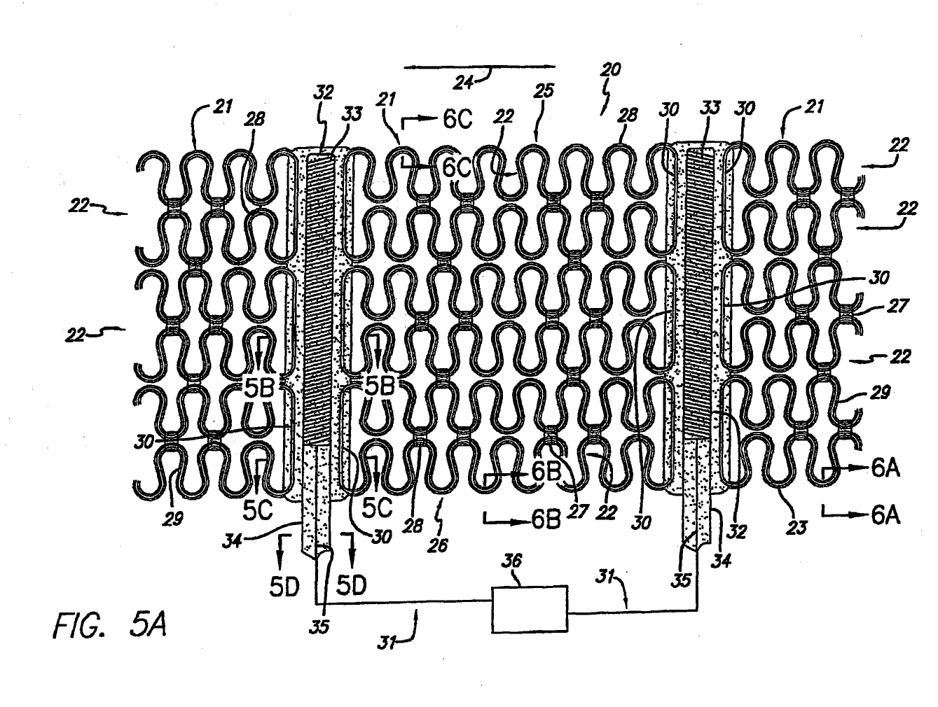

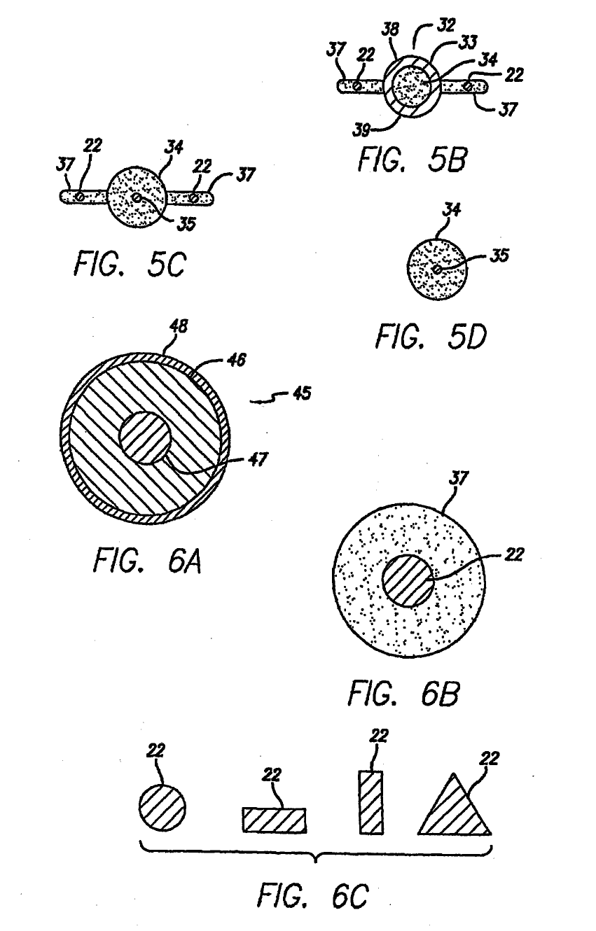

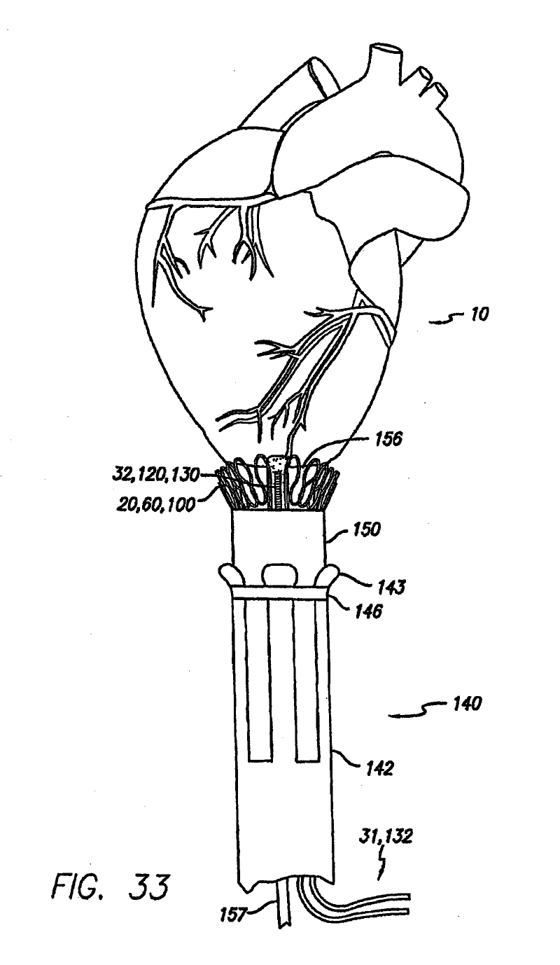

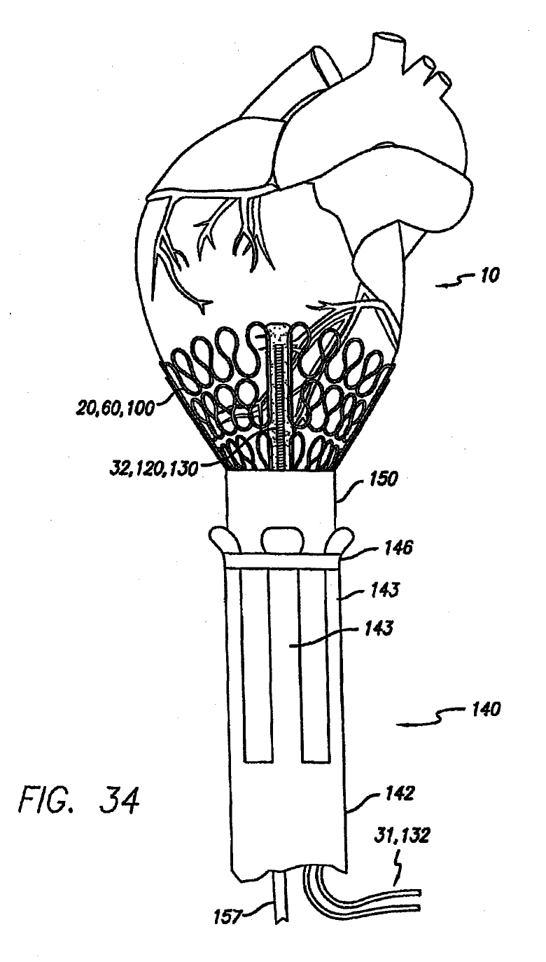

The present invention relates to an apparatus and method of preparing a cardiac harness for use in delivering an mTOR inhibitor or aldosterone blockade from the surface of the cardiac harness to a patient's heart. The present invention discloses embodiments and methods for drug delivery that extend the efficacy of the cardiac harness and the beneficial medicament for use in the site-specific treatment of cardiac and non-cardiac maladies. A cardiac harness is disclosed herein, in FIGS. 5A-42, for use in delivering the mTOR inhibitor or aldosterone blockade. The description for FIGS. 5A-42 precedes the description of the present invention cardiac harness having a drug delivery capability.

### Prior Art Devices

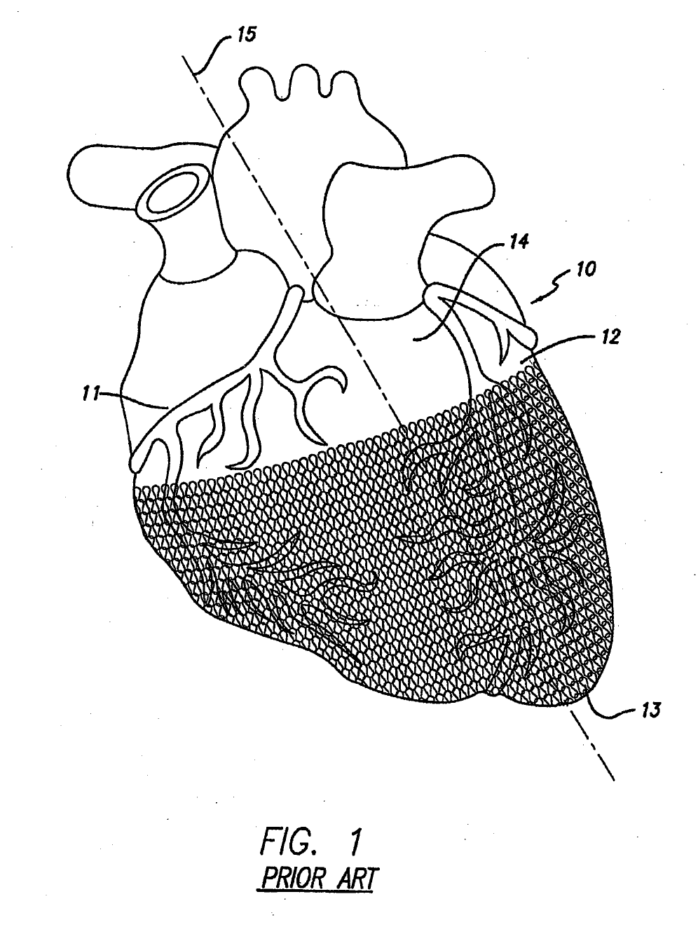

FIG. 1 illustrates a mammalian heart 10 having a prior art cardiac wall stress reduction device in the form of a harness applied to it. The harness surrounds a portion of the heart and covers the right ventricle 11, the left ventricle 12, and the apex 13. For convenience of reference, longitudinal axis 15 goes through the apex and the AV groove 14. The cardiac harness has a series of hinges or spring elements that circumscribe the heart and, collectively, apply a mild compressive force on the heart to alleviate wall stresses.

The term “cardiac harness” as used herein is a broad term that refers to a device fit onto a patient's heart to apply a compressive force on the heart during at least a portion of the cardiac cycle.

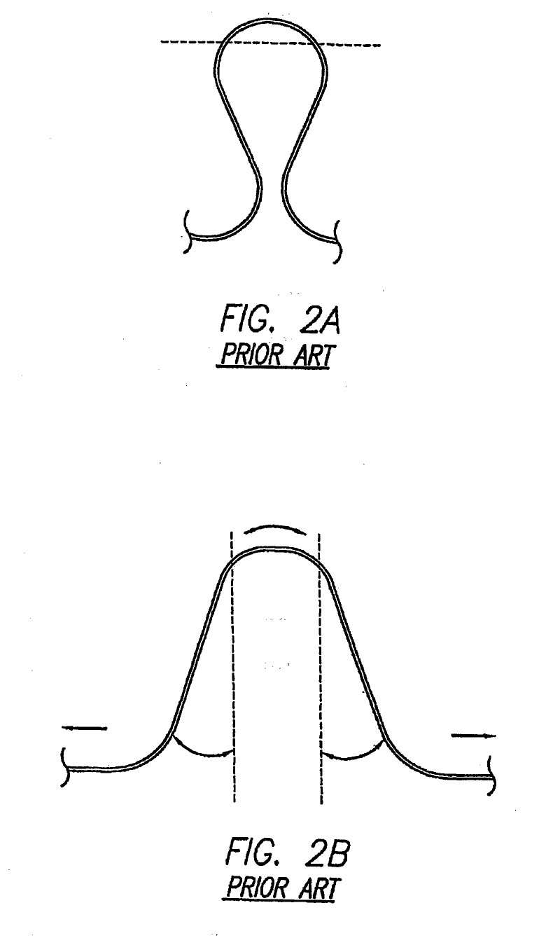

The cardiac harness illustrated in FIG. 1 has at least one undulating strand having a series of spring elements referred to as hinges or spring hinges that are configured to deform as the heart expands during filling. Each hinge provides substantially unidirectional elasticity, in that it acts in one direction and does not provide as much elasticity in the direction perpendicular to that direction. For example, FIG. 2A shows a prior art hinge member at rest. The hinge member has a central portion and a pair of arms. As the arms are pulled, as shown in FIG. 2B, a bending moment is imposed on the central portion. The bending moment urges the hinge member back to its relaxed condition. Note that a typical strand comprises a series of such hinges, and that the hinges are adapted to elastically expand and retract in the direction of the strand.

In the harness illustrated in FIG. 1, the strands of spring elements are constructed of extruded wire that is deformed to form the spring elements.

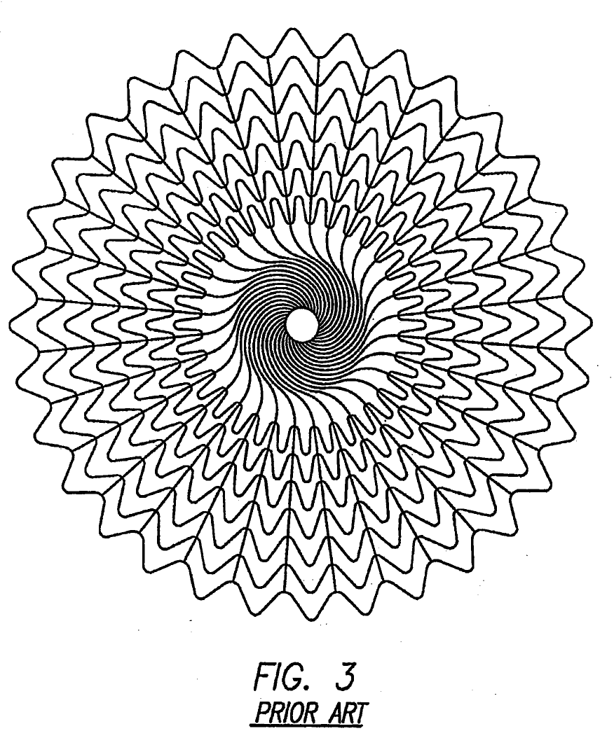

FIGS. 3 and 4 illustrate another prior art cardiac harness, shown at two points during manufacture of such a harness. The harness is first formed from a relatively thin, flat sheet of material. Any method can be used to form the harness from the flat sheet. For example, in one embodiment, the harness is photochemically etched from the material; in another embodiment, the harness is laser-cut from the thin sheet of material. The harness shown in FIGS. 3 and 4 has been etched from a thin sheet of Nitinol, which is superelastic material that also exhibits shape memory properties. The flat sheet of material is draped over a form, die or the like, and is formed to generally take on the shape of at least a portion of a heart.

With further reference to FIGS. 1 and 4, the cardiac harnesses have a base portion which is sized and configured to generally engage and fit onto a base region of a patient's heart, an apex portion which is sized and shaped so as to generally engage and fit on an apex region of a patient's heart, and a medial portion between the base and apex portions.

In the harness shown in FIGS. 3 and 4, the harness has strands or rows of undulating wire. As discussed above, the undulations have hinge/spring elements which are elastically bendable in a desired direction. Some of the strands are connected to each other by interconnecting elements. The interconnecting elements help maintain the position of the strands relative to one another. Preferably the interconnecting elements allow some relative movement between adjacent strands.

The undulating spring elements exert a force in resistance to expansion of the heart. Collectively, the force exerted by the spring elements tends toward compressing the heart, thus alleviating wall stresses in the heart as the heart expands. Accordingly, the harness helps to decrease the workload of the heart, enabling the heart to more effectively pump blood through the patient's body and enabling the heart an opportunity to heal itself. It should be understood that several arrangements and configurations of spring members can be used to create a mildly compressive force on the heart to reduce wall stresses. For example, spring members can be disposed over only a portion of the circumference of the heart or the spring members can cover a substantial portion of the heart.

As the heart expands and contracts during diastole and systole, the contractile cells of the myocardium expand and contract. In a diseased heart, the myocardium may expand such that the cells are distressed and lose at least some contractility. Distressed cells are less able to deal with the stresses of expansion and contraction. As such, the effectiveness of heart pumping decreases. Each series of spring hinges of the above cardiac harness embodiments is configured so that as the heart expands during diastole the spring hinges correspondingly will expand, thus storing expansion forces as bending energy in the spring. As such, the stress load on the myocardium is partially relieved by the harness. This reduction in stress helps the myocardium cells to remain healthy and/or regain health. As the heart contracts during systole, the disclosed prior art cardiac harnesses apply a moderate compressive force as the hinge or spring elements release the bending energy developed during expansion allowing the cardiac harness to follow the heart as it contracts and to apply contractile force as well.

### Cardiac Harness Embodiments For Drug Delivery

Other structural configurations for cardiac harnesses exist, however, but all have drawbacks and do not function optimally to treat CHF and other related diseases or failures. The cardiac harness disclosed herein provides a novel approach to treat CHF and provides electrodes associated with the harness to deliver an electrical shock for defibrillation or a pacing stimulus for resynchronization, or for biventricular pacing/sensing.

A cardiac harness system is disclosed herein for treating the heart. The cardiac harness system couples a cardiac harness for treating the heart coupled with a cardiac rhythm management device. More particularly, the cardiac harness includes rows or undulating strands of spring elements that provide a compressive force on the heart during diastole and systole in order to relieve wall stress pressure on the heart. Associated with the cardiac harness is a cardiac rhythm management device for treating any number of irregularities in heart beat due to, among other reasons, congestive heart failure. Thus, the cardiac rhythm management device associated with the cardiac harness can include one or more of the following: an implantable cardioverter/defibrillator with associated leads and electrodes; a cardiac pacemaker including leads and electrodes used for sensing cardiac function and providing pacing stimuli to treat synchrony of both vessels; and a combined implantable cardioverter/defibrillator and pacemaker, with associated leads and electrodes to provide a defibrillation shock and/or pacing/sensing functions.

The cardiac harness system includes various configurations of panels connected together to at least partially surround the heart and assist the heart during diastole and systole. The cardiac harness system also includes one or more leads having electrodes associated with the cardiac harness and a source of electrical energy supplied to the electrodes for delivering a defibrillating shock or pacing stimuli.

As shown in a flattened configuration in FIG. 5, a cardiac harness 20 includes two panels 21 of generally continuous undulating strands 22. A panel includes rows or undulating strands of hinges or spring elements that are connected together and that are positioned between a pair of electrodes, the rows or undulations being highly elastic in the circumferential direction and, to a lesser extent, in the longitudinal direction. In this embodiment, the undulating strands have U-shaped hinges or spring elements 23 capable of expanding and contracting circumferentially along directional line 24. The cardiac harness has a base or upper end 25 and an apex or lower end 26. The undulating strands are highly elastic in the circumferential direction when placed around the heart 10, and to a lesser degree in a direction parallel to the longitudinal axis 15 of the heart. Similar hinges or spring elements are disclosed in co-pending and co-assigned U.S. Ser. No. 60/458,991 filed Mar. 28, 2003, the entire contents of which are incorporated herein by reference. While the FIG. 5 embodiment appears flat for ease of reference, in use it is substantially cylindrical (of tapered) to conform to the heart and the right and left side panels would actually be one panel and there would be no discontinuity in the undulating strands.

The undulating strands 22 provide a compressive force on the epicardial surface of the heart thereby relieving wall stress. In particular, the spring elements 23 expand and contract circumferentially as the heart expands and contracts during the diastolic and systolic functions. As the heart expands, the spring elements expand and resist expansion as they continue to open and store expansion forces. During systole, as the heart 10 contracts, the spring elements will contract circumferentially by releasing the stored bending forces thereby assisting in both the diastolic and systolic function.

As just discussed, bending stresses are absorbed by the spring elements 23 during diastole and are stored in the elements as bending energy. During systole, when the heart pumps, the heart muscles contract and the heart becomes smaller. Simultaneously, bending energy stored within the spring elements 23 is at least partially released, thereby providing an assist to the heart during systole. The compressive force exerted on the heart by the spring elements of the harness comprises about 10% to 15% of the mechanical work done as the heart contracts during systole. Although the harness is not intended to replace ventricular pumping, the harness does substantially assist the heart during systole.

The undulating strands 22 can have varying numbers of spring element 23 depending upon the amplitude and pitch of the spring elements. For example, by varying the amplitude of the pitch of the spring elements, the number of undulations per panel will vary as well. It may be desired to increase the amount of compressive force the cardiac harness 20 imparts on the epicardial surface of the heart, therefore panels that have spring elements with lower amplitudes and a shorter pitch, thereby increasing the expansion force imparted by the spring element, are disclosed. In other words, all other factors being constant, a spring element having a relatively lower amplitude will be more rigid and resist opening, thereby storing more bending forces during diastole. Further, if the pitch is smaller, there will be more spring elements per unit of length along the undulating strand, thereby increasing the overall bending force stored during diastole, and released during systole. Other factors that will affect the compressive force imparted by the cardiac harness onto the epicardial surface of the heart include the shape of the spring elements, the diameter and shape of the wire forming the undulating strands, and the material comprising the strands.

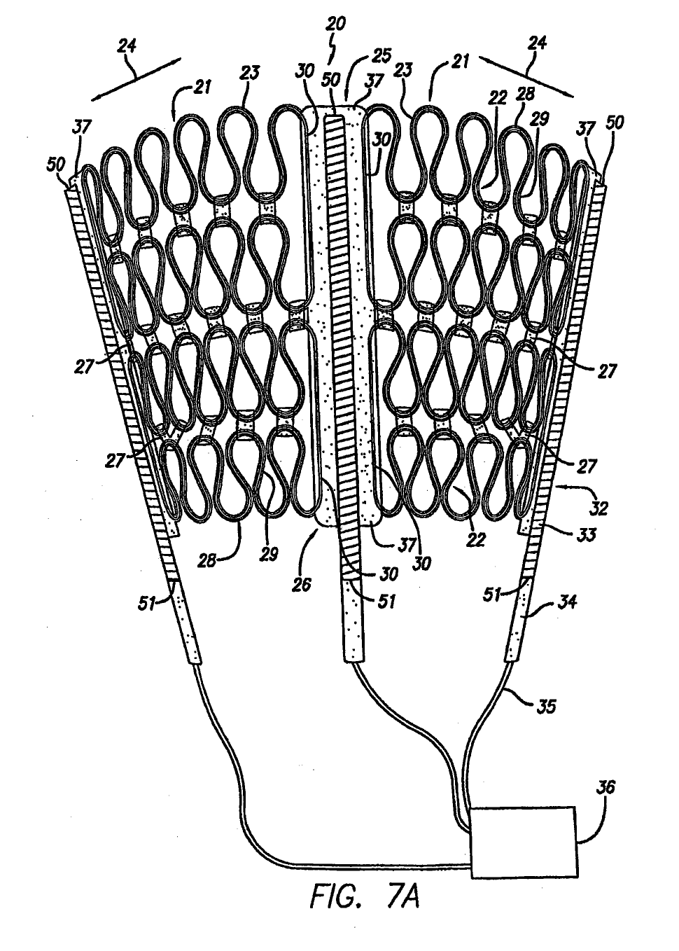

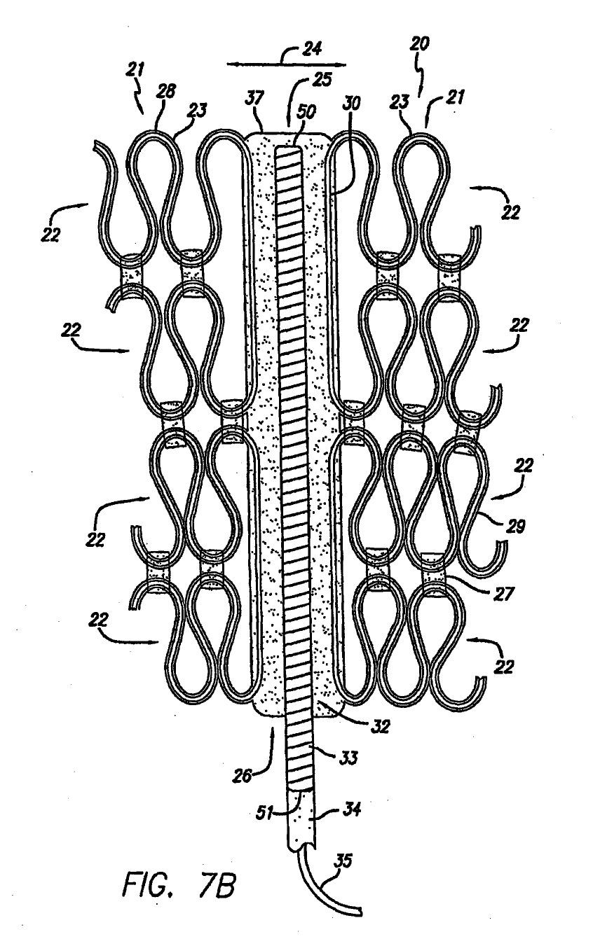

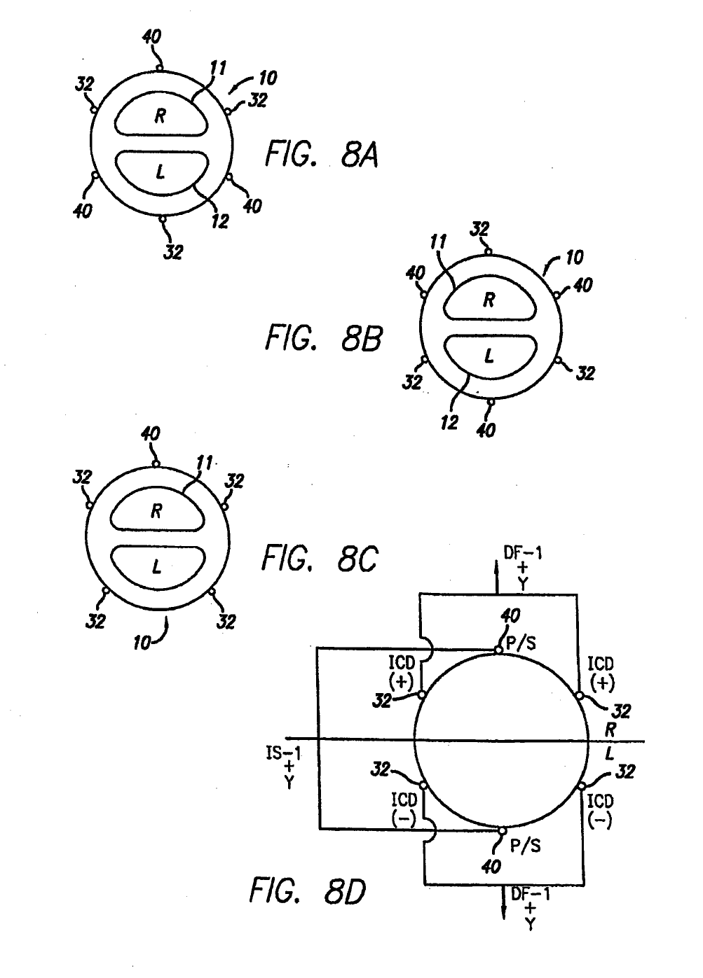

As shown in FIG. 5, the undulating strands 22 are connected to each other by grip pads 27. In the embodiments shown in FIG. 5, adjacent undulating strands are connected by one or more grip pads attached at the apex 28 of the spring elements 23. The number of grip pads between adjacent undulating strands is a matter of choice and can range from one grip pad between adjacent undulating strands, to one grip pad for every apex on the undulating strand. Importantly, the grip pads should be positioned in order to maintain flexibility of the cardiac harness 20 without sacrificing the objectives of maintaining the spacing between adjacent undulating strands to prevent overlap and to enhance the frictional engagement between the grip pads and the epicardial surface of the heart. Further, while it is desirable to have the grip pads attached at the apex of the spring elements, it is not necessary. The grip pads 27 can be attached anywhere along the length of the spring elements, including the sides 29. Further, the shape of the grip pads 27, as shown in FIG. 5, can vary to suit a particular purpose. For example, grip pad 27 can be attached to the apex 28 of one undulating strand 22, and be attached to two apices on an adjacent undulating strand (see FIG. 7). As shown in FIG. 5, all of the apices point toward each other, and are said to be “out-of-phase.” If the apices of the undulations were aligned, they would be “in-phase.” The apices are all out-of-phase since the number of spring elements in each undulating strand is the same, however, it is possible that the number of spring elements in each undulating strand may vary since the heart is tapered from its base near the top to its apex 13 at the bottom. Thus, there would be more spring elements and a longer undulating strand per panel at the top or base of the cardiac harness than at the bottom of the cardiac harness near the apex of the heart. Accordingly, the cardiac harness would be tapered from the relatively wide base to a relatively narrow bottom toward the apex of the heart, and this would affect the alignment of the apices of the spring elements, and hence the ability of the grip pads 27 to align perfectly and attach to adjacent apices of the spring elements. A further disclosure and embodiments relating to the undulating strands and the attachment means in the form of grip pads is found in co-pending and co-assigned U.S. Ser. No. 60/486,062 filed Jul. 10, 2003, now U.S. Ser. No. 10/888,806 filed Jul. 8, 2004, the entire contents of which are incorporated herein by reference. While the connections between adjacent undulating strands 22 is preferably grip pads 27, alternatively, the undulating strands are connected by interconnecting elements made of the same material as the strands. The interconnecting elements can be straight or curved as shown in FIGS. 8A 8B of commonly owned U.S. Pat. No. 6,612,979 B2, the entire contents of which is incorporated by reference herein.

It is preferred that the undulating strands 22 be continuous as shown in FIG. 5. For example, every pair of adjacent undulating strands are connected by bar arm 30. It is preferred that the bar arms form part of a continuous wire that is bent to form the undulating strands, and then welded at its ends along the bar arm. The weld is not shown in FIG. 5, but can be by any conventional method such as laser welding, fusion bonding, or conventional welding. The type of wire used to form the undulating strands may have a bearing on the method of attaching the ends of the wire used to form the undulating strand. For example, it is preferred that the undulating strands be made out of a nickel-titanium alloy, such as Nitinol, which may lose some of its superelastic or shape memory properties if exposed to high heat during conventional welding.

Associated with the cardiac harness is a cardiac rhythm management device as previously disclosed. Thus, associated with the cardiac harness as shown in FIG. 5, are one or more electrodes for use in providing defibrillating shock. As can be seen immediately below, any number of factors associated with congestive heart failure can lead to fibrillation, acquiring immediate action to save the patient's life.

Diseased hearts often have several maladies. One malady that is not uncommon is irregularity in heartbeat caused by irregularities in the electrical stimulation system of the heart. For example, damage from a cardiac infarction can interrupt the electrical signal of the heart. In some instances, implantable devices, such as pacemakers, help to regulate cardiac rhythm and stimulate heart pumping. A problem with the heart's electrical system can sometimes cause the heart to fibrillate. During fibrillation, the heart does not beat normally, and sometimes does not pump adequately. A cardiac defibrillator can be used to restore the heart to normal beating. An external defibrillator typically includes a pair of electrode paddles applied to the patient's chest. The defibrillator generates an electric field between electrodes. An electric current passes through the patient's heart and stimulates the heart's electrical system to help restore the heart to regular pumping.

Sometimes a patient's heart begins fibrillating during heart surgery or other open-chest surgeries. In such instances, a special type of defibrillating device is used. An open-chest defibrillator includes special electrode paddles that are configured to be applied to the heart on opposite sides of the heart. A strong electric field is created between the paddles, and an electric current passes through the heart to defibrillate the heart and restore the heart to regular pumping.

In some patients that are especially vulnerable to fibrillation, an implantable heart defibrillation device may be used. Typically, an implantable heart defibrillation device includes an implantable cardioverter defibrillator (ICD) or a cardiac resynchronization therapy device (CRT-D) which usually has only one electrode positioned in the right ventricle, and the return electrode is the defibrillator housing itself, typically implanted in the pectoral region. Alternatively, an implantable device includes two or more electrodes mounted directly on, in or adjacent the heart wall. If the patient's heart begins fibrillating, these electrodes will generate an electric field therebetween in a manner similar to the other defibrillators discussed above.

Testing has indicated that when defibrillating electrodes are applied external to a heart that is surrounded by a device made of electrically conductive material, at least some of the electrical current disbursed by the electrodes is conducted around the heart by the conductive material, rather than through the heart. Thus, the efficacy of defibrillation is reduced. Accordingly, there are several cardiac harness embodiments that enable defibrillation of the heart and other embodiments disclose means for defibrillating, resynchronization, left ventricular pacing, right ventricular pacing, and biventricular pacing/sensing.

The cardiac harness 20 includes a pair of leads 31 having conductive electrode portions 32 that are spaced apart and which separate panels 21. As shown in FIG. 5, the electrodes are formed of a conductive coil wire 33 that is wrapped around a non-conductive member 34, preferably in a helical manner. A conductive wire 35 is attached to the coil wire and to a power source 36. The power source 36 can include any of the following, depending upon the particular application of the electrode: a pulse generator; an implantable cardioverter/defibrillator; a pacemaker; and an implantable cardioverter/defibrillator coupled with a pacemaker. In the embodiment shown in FIG. 5, the electrodes are configured to deliver an electrical shock, via the conductive wire and the power source, to the epicardial surface of the heart so that the electrical shock passes through the myocardium. Even though the electrodes are spaced so that they would be about 180 degrees apart around the circumference of the heart in the embodiment shown, they are not so limited. In other words, the electrodes can be spaced so that they are about 45 degrees apart, 60 degrees apart, 90 degrees apart, 120 degrees apart, or any arbitrary arc length spacing, or, for that matter, essentially any arc length apart around the circumference of the heart in order to deliver an appropriate electrical shock. As previously described, it may become necessary to defibrillate the heart and the electrodes 32 are configured to deliver an appropriate electrical shock to defibrillate the heart.

Still referring to FIG. 5, the electrodes 32 are attached to the cardiac harness 20, and more particularly to the undulating strands 22, by a dielectric material 37. The dielectric material insulates the electrodes from the cardiac harness so that electrical current does not pass from the electrode to the harness thereby undesirably shunting current away from the heart for defibrillation. The dielectric material may cover the undulating strands 22 and covers at least a portion of the electrodes 32. In the FIG. 5 embodiment, the middle panel undulating strands are covered with the dielectric material while the right and left side panels are bare metal. While it is preferred that all of the undulating strands of the panels be coated with the dielectric material, thereby insulating the harness from the electric shock delivered by the electrodes, some or all of the undulating strands can be bare metal used to deliver the electrical shock to the epicardial surface of the heart for defibrillation or for pacing.

As will be described in more detail, the electrodes 32 have a conductive discharge first surface 38 that is intended to be proximate to or in direct contact with the epicardial surface of the heart, and a conductive discharge second surface 39 that is opposite to the first surface and faces away from the heart surface. As used herein, the term “proximate” is intended to mean that the electrode is positioned near or in direct contact with the outer surface of the heart, such as the epicardial surface of the heart. The first surface and second surface typically will not be covered with the dielectric material 37 so that the bare metal conductive coil can transmit the electrical current from the power source (pulse generator), such as an implantable cardioverter/defibrillator (ICD or CRT-D) 36, to the epicardial surface of the heart. Alternatively, either the first or the second surface may be covered with dielectric material 37 in order to preferentially direct the current through only one surface.

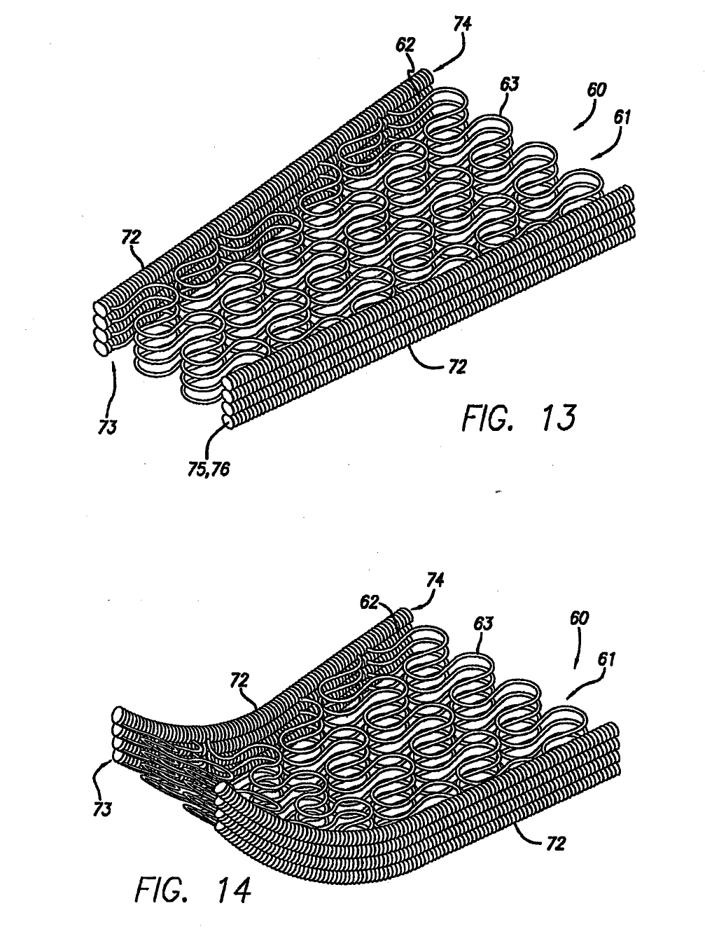

Importantly, the dielectric material 37 used to attach the electrodes 32 to the undulating strands 22 insulates the undulating strands from any electrical current discharged through the conductive metal coils 33 of the electrodes. Further, the dielectric material in this embodiment is flexible so that the electrodes can serve as a seam or hinge to fold the cardiac harness 20 into a lower profile for minimally invasive delivery. Thus, as will be described in more detail (see FIGS. 13 and 14), the cardiac harness can be folded along its length, along the length of the electrodes, in order to reduce the profile for intercostal delivery, for example through the rib cage or other area typically used for minimally invasive surgery for accessing the heart. Minimally invasive approaches involving the heart typically are made through subxiphoid, subcostal or intercostal incisions. When the cardiac harness is folded, it can be reduced into a circular or a more or less oval shape, both of which promote minimally invasive procedures.

Cross sectional views of the leads 31 and the electrode portion 32 are shown in FIGS. 5B, 5C, and 5D. As shown in FIG. 5B, the electrode 32 has the coil wire 33 wrapped around the non-conducting member 34 in a helical pattern. The dielectric material 37 provides a spaced connection between the electrode and the bar arms 30 at the ends of the undulating strands 22. The electrodes do not touch or overlap with the bar arms or any portion of the undulating strands. Instead, the dielectric material provides the attachment means between the electrodes and the bar arms of the undulating strands. Thus, the dielectric material 37 not only acts as an insulating non-conductive material, but also provides attachment means between the undulating strands and the electrodes. Because the dielectric material 37 is relatively thin at the attachment points, it is highly flexible and permits the electrodes to be flexible along with the cardiac harness panels 21, which will expand and contract as the heart beats as previously described.

Referring to FIG. 5C, the non-conductive member 34 extends beyond the coil wire 33 for a distance. The non-conductive member preferably is made from the same material as the dielectric material 37, typically a silicone rubber or similar material. While it is preferred that the dielectric material be made from silicone rubber, or a similar material, it also can be made from Parylene™ (Union Carbide), polyurethanes, PTFE, TFE, and ePTFE. As can be seen, the non-conductive member provides support for the dielectric material to attach the bar arms 30 of the undulating strands 22 in order to connect the strands to the electrode 32. A conductive wire 35 extends through the non-conducting member and attaches to the proximal end of the coil wire 33 so that when an electrical current is delivered from the power source 36 through conductive wire 35, the electrode coil 33 will be energized. The conductive wire 35 is also covered by non-conducting material 34. Referring to FIG. 5D, it can be seen that the non-conductive member 34 continues to extend beyond the bottom (apex) of the cardiac harness and that conductive wire 35 continues to extend out of the non-conductive member and into the power source 36. In the embodiment shown in FIGS. 5B 5D, the cardiac harness is insulated from the electrodes by the dielectric material 37 so that there is no shunting of electrical currents by the cardiac harness 20 from the electrical shock delivered by the electrodes during defibrillation or pacing functions.

While it is preferred that the cardiac harness 20 be comprised of undulating strands 22 made from a solid wire member, such as a superelastic or shape memory material such as Nitinol, and be insulated from the electrodes 32, it is possible to use some or all of the undulating strands to deliver the electrical shock to the epicardial surface of the heart. For example, as shown in FIG. 6A, a composite wire 45 can be used to form the undulating strands 22 and, importantly, to effectively transmit current to deliver an electrical shock to the epicardial surface of the heart. The composite wire 45 includes a current conducting wire 47 made from, for example silver (Ag), and which is covered by a Nitinol tube 46. In order to improve the surface conductivity of the outer Nitinol tube 46, a highly conductive coating is placed on the Nitinol tube. For example, the Nitinol tube can be covered with a deposition layer of platinum (Pt) or platinum-iridium (Pt—Ir), or an equivalent material including iridium oxide (IROX). The composite wire, so constructed, will have superior mechanical performance to expand and contract due to the Nitinol tubing, and also will have improved electrical properties resulting from the current conducting wire 47 and improved electrolytic/electrochemical properties via the surface layer of platinum-iridium. Thus, if some portion or all of the undulating strands 22 are made from a composite wire 45, the cardiac harness 20 will be capable of delivering a defibrillating shock on selected portions of the heart via the undulating strands and will also function to impart compressive forces as previously described.

In contrast to the current conducting undulating strands of FIG. 6A, are the non-conducting insulated undulating strands 22 as shown by cross sectional view FIG. 6B. As previously described, some or all of the undulating strands 22 can be covered with dielectric material 37 in order to insulate the strands from the electrical current delivered through the electrodes while delivering shock on the epicardial surface of the heart. Thus, as shown in FIG. 6B, the undulating strands 22 are covered by dielectric material 37 to provide insulation from the electrical shock delivered by the electrodes 32, yet maintain the flexibility and the expansive properties of the undulating strands.

A cardiac harness 20 that can be implanted minimally invasively and be attached to the epicardial surface of the heart, without requiring sutures, clips, screws, glue or other attachment means, is provided. Importantly, the undulating strands 22 may provide relatively high frictional engagement with the epicardial surface, depending on the cross-sectional shape of the strands. For example, in the embodiment disclosed in FIG. 6C, the cross-sectional shape of the undulating strands 22 can be circular, rectangular, triangular or for that matter, any shape that increases the frictional engagement between the undulating strands and the epicardial surface of the heart. As shown in FIG. 6C, the middle cross-section view having a flat rectangular surface (wider than tall) not only has a low profile for enhancing minimally invasive delivery of the cardiac harness, but it also has rectangular edges that may have a tendency to engage and dig into the epicardium to increase the frictional engagement with the epicardial surface of the heart. With the proper cross-sectional shape for the undulating strands, coupled with the grip pads 27 having a high frictional engagement feature, the necessity for suturing, clipping, or further attachment means to attach the cardiac harness to the epicardial surface of the heart becomes unnecessary.

In another embodiment as shown in FIGS. 7A and 7B, a different configuration for cardiac harness 20 and the electrodes 32 are shown, as compared to the FIG. 5 embodiments. In FIGS. 7A and 7B, three electrodes are shown separating the three panels 21 with undulating strands 22 extending between the electrodes. As with previous embodiments, springs 23 are formed by the undulating strands so that the undulating strands can expand and contract during the diastolic and systolic functions, and apply a compressive force during both functions. The far side panel of FIG. 7A is not shown for clarity purposes. The position of the electrodes around the circumference of the heart is a matter of choice, and in the embodiment of FIG. 7A, the electrodes can be spaced an equal distance apart at about 120 degrees. Alternatively, it may be important to deliver the electrical shock more through the right ventricle requiring the positioning of the electrodes closer to the right ventricle than to the left ventricle. Similarly, it may be more important to deliver an electrical shock to the left ventricle as opposed to the right ventricle. Thus, positioning of electrodes, as with other embodiments, is a matter of choice.

Still referring to FIGS. 7A and 7B, electrodes 32 extend beyond the bottom or apex portion of the cardiac harness 20 in order to insure that the electrical shock delivered by the electrodes is delivered to the epicardial surface of the heart and including the lower portion of the heart closer to the apex 13. Thus, the electrodes 22 have a distal end 50 and a proximal end 51 where the proximal end is positioned closer to the apex 13 of the heart and the distal end is positioned closer to the base or upper portion of the heart. As used herein, distal is intended to mean further into the body and away from the attending physician, and proximal is meant to be closer to the outside of the body and closer to the attending physician. The proximal ends of the electrodes are positioned closer to the apex of the heart and provide several functions, including the ability to deliver an electrical shock closer to the apex of the heart. The electrode proximal ends also function to provide support for the cardiac harness 20 and the panels 21, and lend support not only during delivery (as will be further described herein) but in separating the panels and in gripping the epicardial surface of the heart to retain the harness on the heart without slipping.

While the FIGS. 7A and 7B embodiments show electrodes 32 separating three panels 21 of the cardiac panel 20, more or fewer electrodes and panels can be provided to suit a particular application. For example, four electrodes 32 separate four panels 21, so that two of the electrodes can be positioned on opposite sides of the left ventricle and two of the electrodes can be positioned on opposite sides of the right ventricle. Preferably all four electrodes would be used, with a first set of two electrodes on opposite sides of the right ventricle acting as one (common) electrode and a second set of two electrodes on opposite sides of the left ventricle acting as the opposite (common) electrode. Alternatively, two of the electrodes can be activated while the other two electrodes act as dummy electrodes in that they would not be activated unless necessary.

At present, commercially available implantable cardioverter/defibrillators (ICD's) are capable of delivering approximately thirty to forty joules in order to defibrillate the heart. It is preferred that the electrodes 22 of the cardiac harness 20 of the present invention deliver defibrillating shocks having less than thirty to forty joules. The commercially available ICD's can be modified to provide lower power levels to suit the present invention cardiac harness system with electrodes delivering less than thirty to forty joules of power. As a general rule, one objective of the electrode configuration is to create a uniform current density distribution throughout the myocardium. Therefore, in addition to the number of electrodes used, their size, shape, and relative positions will also all have an impact on the induced current density distribution. Thus, while one to four electrodes are preferred, five to eight electrodes also are feasible.

The cardiac harness and the associated cardiac rhythm management device can be used not only for providing a defibrillating shock, but also can be used as a pacing/sensing device for treating the synchrony of both ventricles, for resynchronization, for biventricular pacing and for left ventricular pacing or right ventricular pacing. As shown in FIGS. 8A 8D, the heart 10 is shown in cross-section exposing the right ventricle 11 and the left ventricle 12. The cardiac harness 20 is mounted around the outer surface of the heart, preferably on the epicardial surface of the heart, and multiple electrodes are associated with the cardiac harness. More specifically, electrodes 32 are attached to the cardiac harness and positioned around the circumference of the heart on opposite sides of the right and left ventricles. In the event that fibrillation should occur, the electrodes will provide an electrical shock through the myocardium and the left and right ventricles in order to defibrillate the heart. Also mounted on the cardiac harness, is a pacing/sensing lead 40 that functions to monitor the heart and provide data to a pacemaker. If required, the pacemaker will provide pacing stimuli to synchronize the ventricles, and/or provide left ventricular pacing, right ventricular pacing or biventricular pacing. Thus, for example, in FIG. 8C, pairs of pacing/sensing leads 40 are positioned adjacent the left and right ventricle free walls and can be used to provide pacing stimuli to synchronize the ventricles, or provide left ventricular pacing, right ventricular pacing or biventriculator pacing. The use of proximal Y connectors can simplify the transition to a post-generator such as Oscor's, iLink-B15-10. The iLink-B15-10 can be used to link the right and left ventricular free-wall pace/sense leads 40, as shown in 8D.

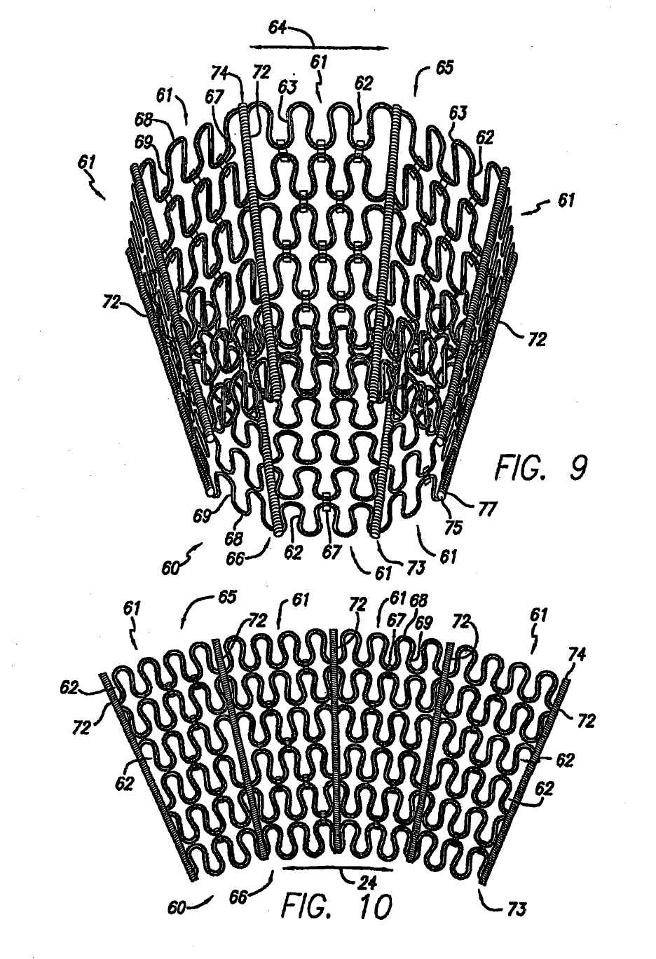

As shown in FIGS. 9-14, cardiac harness 60 is similar to previously described cardiac harness 20. With respect to cardiac harness 60, it also includes panels 61 consisting of undulating strands 62. The undulating strands are continuous and extend through coils as will be described. The undulating strands act as spring elements 63 as with prior embodiments that will expand and contract along directional line 64. The cardiac harness 60 includes a base or upper end 65 and an apex or lower end 66. In order to add stability to the cardiac harness 60, and to assist in maintaining the spacing between the undulating strands 62, grip pads 67 are connected to adjacent strands, preferably at the apex 68 of the springs. Alternatively, the grip pads 67 could be attached from the apex of one spring element to the side 69 of a spring element, or the grip pad could be attached from the side of one spring to the side of an adjacent spring on an adjacent undulating strand. As shown in the FIGS. 9-14, in order to add stability and some mechanical stiffness to the cardiac harness 60, coils 62 are interwoven with the undulating strands, which together define the panels 61. The coils typically are formed of a coil of wire such as Nitinol or similar material (stainless steel, MP35N), and are highly flexible along their longitudinal length. The coils 72 have a coil apex 73 and a coil base 74 to coincide with the harness base 65 and the harness apex 66. The coils can be injected with a non-conducting material so that the undulating strands extend through gaps in the coils and through the non-conducting material. The non-conducting material also fills in the gaps which will prevent the undulating strands from touching the coils so there is no metal-to-metal touching between the undulating strands and the coils. Preferably, the non-conducting material is a dielectric material 77 that is formed of silicone rubber or equivalent material as previously described. Further, a dielectric material 78 also covers the undulating strands in the event a defibrillating shock or pacing stimuli is delivered to the heart via an external defibrillator (e.g., transthoracic) or other means.

Importantly, coils 72 not only perform the function of being highly flexible and provide the attachment means between the coils and the undulating strands, but they also provide structural columns or spines that assist in deploying the harness 60 over the epicardial surface of the heart. Thus, as shown for example in FIG. 12, the cardiac harness 60 has been positioned over the heart and delivered by minimally invasive means, as will be described more fully herein. The coils 72, although highly flexible along their longitudinal length, have sufficient column strength in order to push on the apex 73 of the coils so that the base portion 74 of the coils and of the harness 65 slide over the apex of the heart and along the epicardial surface of the heart until the cardiac harness 60 is positioned over the heart, substantially as shown in FIG. 12.

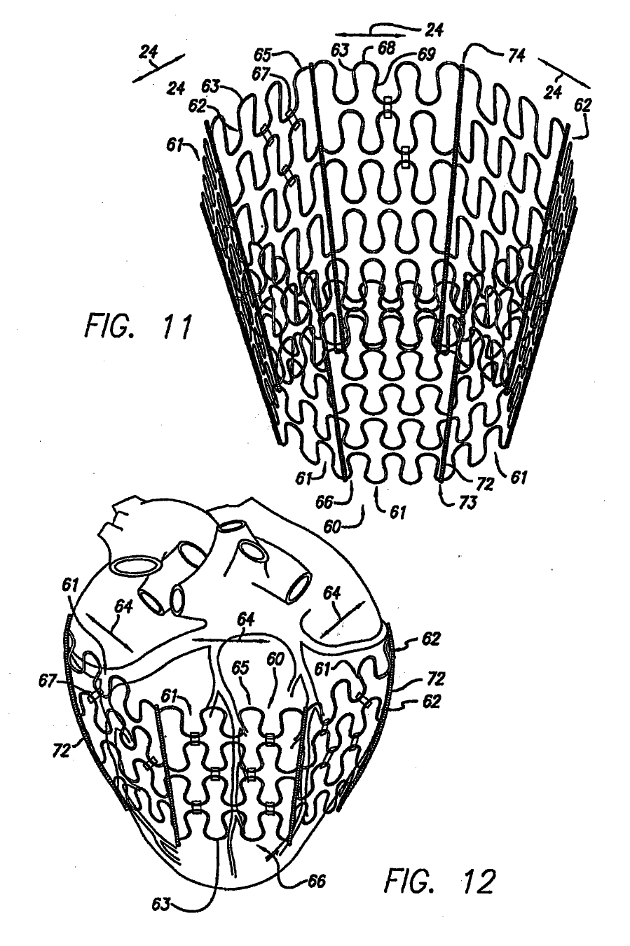

Referring to the embodiments shown in FIGS. 9 and 11, the cardiac harness 60 has multiple panels 61 and multiple coils 72. More or fewer panels and coils can be used in order to achieve a desired result. For example, eight coils are shown in FIGS. 9 and 11, while fewer coils may provide a harness with greater flexibility since the undulating strands 62 would be longer in the space between each coil. Further, the diameter of the coils can be varied in order to increase or decrease flexibility and/or column strength in order to assist in the delivery of the harness over the heart. The coils preferably have a round cross-sectional wire in the form of a tightly wound spiral or helix so that the cross-section of the coil is circular. However, the cross-sectional shape of the coil need not be circular, but may be more advantageous if it were oval, rectangular, or another shape. Thus, if coils 72 had an oval shape, where the longer axis of the oval was parallel to the circumference of the heart, the coil would flex along its longitudinal axis and still provide substantial column strength to assist in delivery of the cardiac harness 60. Further, an oval-shaped coil would provide a lower profile for minimally invasive delivery. The wire cross-section also need not be round/circular, but can consist of a flat ribbon having a rectangular shape for low profile delivery. The coils also can have different shapes, for example they can be closed coils, open coils, laser-cut coils, wire-wound coils, multi-filar coils, or the coil strands themselves can be coiled (i.e., coiled coils). The electrode need not have a coil of wire, rather the electrode could be formed by a zig-zag-shaped wire (not shown) extending along the electrode. Such a design would be highly flexible and fatigue resistant yet still be capable of providing a defibrillating shock.

The cardiac harness embodiments 60 shown in FIGS. 9-12, can be folded as shown in FIGS. 13 and 14 and yet remain highly flexible for minimally invasive delivery. The coils 72 act as hinges or spines so that the cardiac harness can be folded along the longitudinal axis of the coils. The grip pads typically connecting adjacent undulating strands 62 have been omitted for clarity in these embodiments, however, they would be used as previously described.

Similar to the embodiment shown in FIGS. 9 12, the cardiac harness 60 includes both coils 72 and electrodes 32. In this embodiment, as with the previously described embodiments, a series of undulating strands 22 extend between the coils and the electrodes to form panels 21. For example, the coils and electrodes form hinge regions so that the panels can be folded along the longitudinal axis of the coils and electrodes for minimally invasive delivery. Further, there are two coils and four electrodes, with two of the electrodes positioned adjacent the right ventricle, with the remaining two electrodes being positioned adjacent the left ventricle. The coils not only act as a hinge, but provide column strength as previously described so that the cardiac harness can be delivered minimally invasively by delivery through, for example, the intercostal space between the ribs and then pushing the harness over the heart. Likewise, the electrodes provide column strength as well, yet remain flexible along their longitudinal axis, as do the coils.

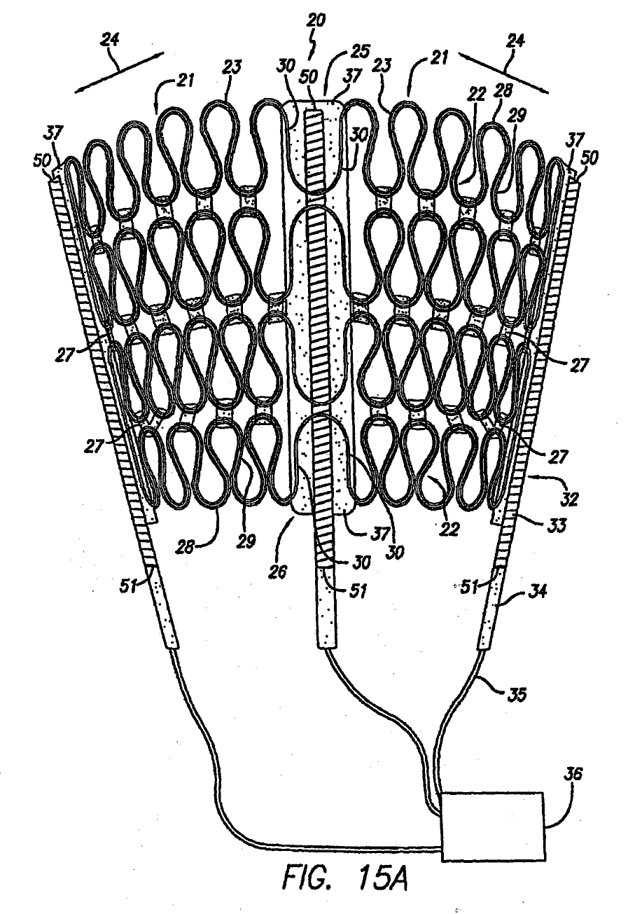

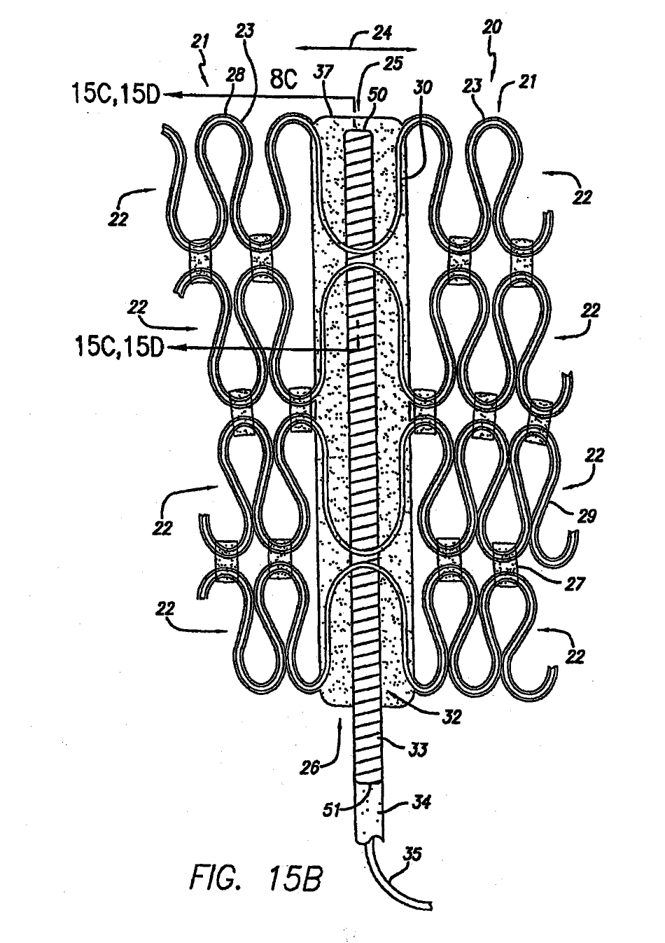

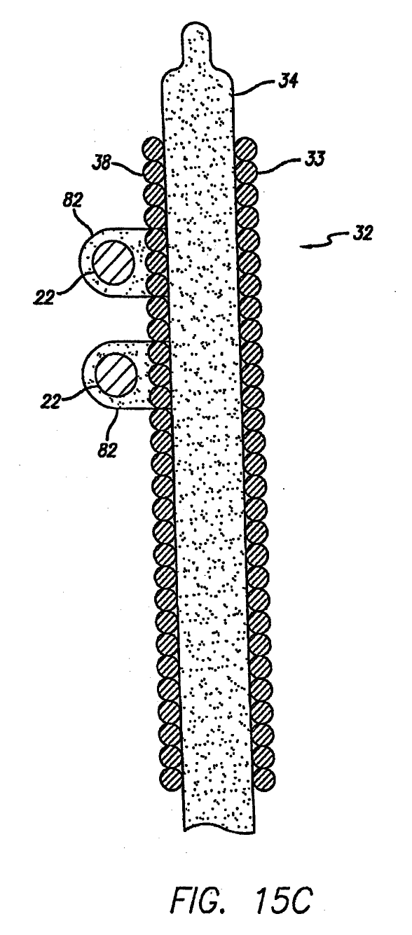

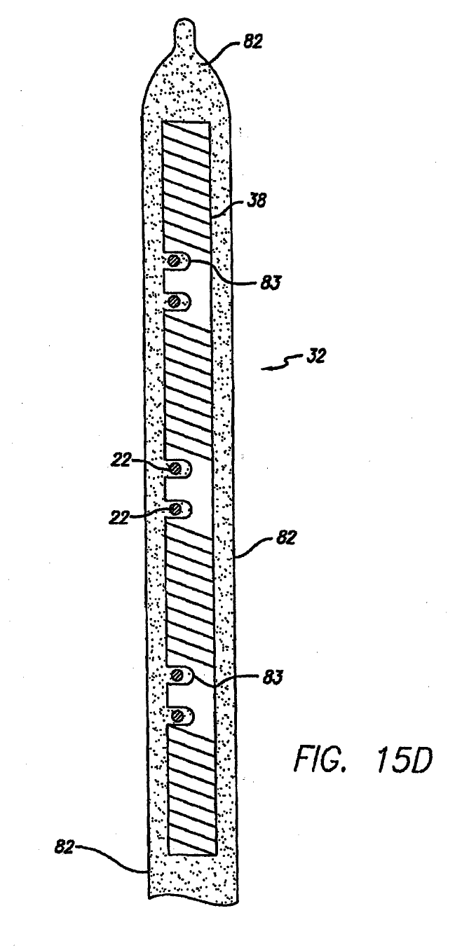

Referring to FIGS. 15A-15D, the electrodes 32 or the coils 72 can be mounted on the inner surface (touching the heart) or outer surface (away from the heart) of the cardiac harness. Thus, the cardiac harness 20 includes continuous undulating strands 22 that extend circumferentially around the heart without any interruptions. The undulating strands are interconnected by any interconnecting means, including grip pads 27, as previously described. In this embodiment, electrodes 32 or coils 72, or both, are mounted on an inner surface 80 or an outer surface 81 of the cardiac harness 20. A dielectric material 82 is molded around the electrodes or coils and around the undulating strands in order to connect the electrodes and coils to the cardiac harness. Alternatively, as shown in FIG. 15D, the electrodes 32 or coils 72 can be formed into a fastening means by forming notches 83 into the electrode (or coil) and which are configured to receive portions of the undulating strand 22. The undulating strands 22 are spaced from the coils or electrodes so that there is no overlapping/touching of metal. The notches 83 are filled with a dielectric material, preferably silicone rubber, or similar material that insulates the undulating strands of the cardiac harness from the electrodes yet provides a secure attachment means so that the electrodes or coils remain firmly attached to the undulating strands of the cardiac harness. Importantly, the electrodes 32 do not have to be in contact with the epicardial surface of the heart in order to deliver a defibrillating shock. Thus, the electrodes 32 can be mounted on the outer surface 81 of the cardiac harness, and not be in physical contact with the epicardial surface of the heart, yet still deliver a defibrillating shock as previously described.

It is to be understood that several embodiments of cardiac harnesses can be constructed and that such embodiments may have varying configurations, sizes, flexibilities, etc. Such cardiac harnesses can be constructed from many suitable materials including various metals, fabrics, plastics and braided filaments. Suitable materials also include superelastic materials and materials that exhibit shape memory properties. For example, a preferred embodiment cardiac harness is constructed of Nitinol. Shape memory dielectric materials can also be employed. Such shape memory dielectric materials can include shape memory polyurethanes or other dielectric materials such as those containing oligo(e-caprolactone) dimethacrylate and/or poly(e-caprolactone), which are available from mnemoScience.

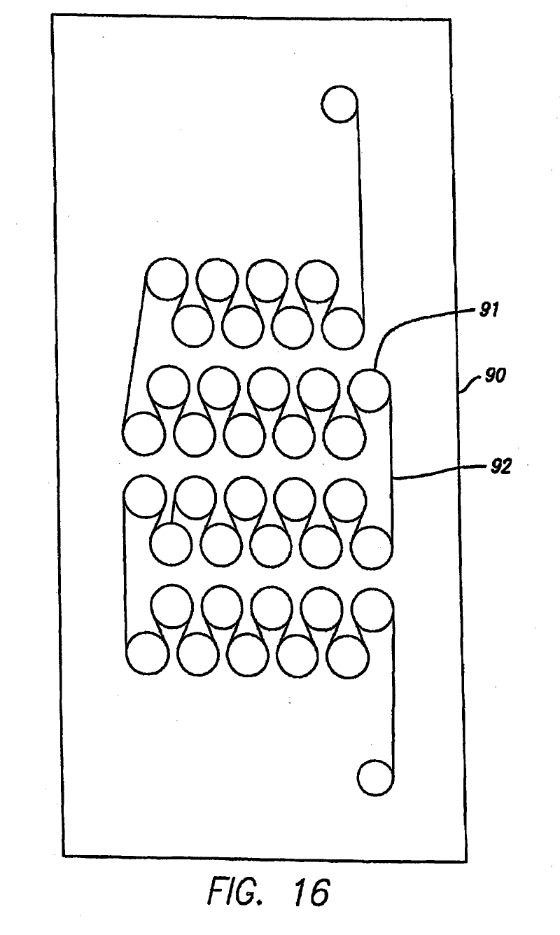

As shown in FIG. 16, the undulating strands 22 and 62 can be formed in many ways, including by a fixture 90. The fixture 90 has a number of stems 91 that are arranged in a pre-selected pattern that will define the shape of the undulating strands 22 and 62. The position of the stems will define the shape of the undulating strands, and determine whether the previously disclosed apex of the springs is either in-phase or out-of-phase. The shape of stems 91 will define the shape of the springs in terms of radius of curvature, or other shape, such as a keyhole shape, a U-shape, and the like. The spacing between the stems will determine the pitch and the amplitude of the undulating strands which is a matter of choice. Preferably, in one exemplary embodiment, a Nitinol wire 92 or other superelastic or shape memory wire having a 0.012 inch diameter, is woven between stems 91 in order to form the undulating strands. Other wire diameters can be used to suit a particular need and can range from about 0.007 inch to about 0.020 inch diameter. Other wire cross-section shapes are envisioned to be used with fixture 90, particularly a flat rectangular-shaped wire and an oval-shaped wire. The Nitinol wire is then heat set to impart the shape memory feature. Any free ends can be connected together by laser bonding, laser welding, or other type of similar connection consistent with the use of Nitinol, or the ends may remain free and be encapsulated in a dielectric material to keep them atraumatic, depending upon the design.

Again referring to FIG. 16, after the Nitinol wire is heat set to impart the shape memory feature, the wire is jacketed with NuSil silicone tubing (Helix Medical) having 0.029 inch outside diameter by 0.012 inch inside diameter. Thereafter, the jacketed Nitinol wire is placed in molds for transfer of liquid silicone rubber which will insulate the Nitinol wire from any electrical shock from any electrodes associated with the cardiac harness, or any other device providing a defibrillating shock to the heart. The dimensions of the silicone tubing will of course vary for different wire dimensions.

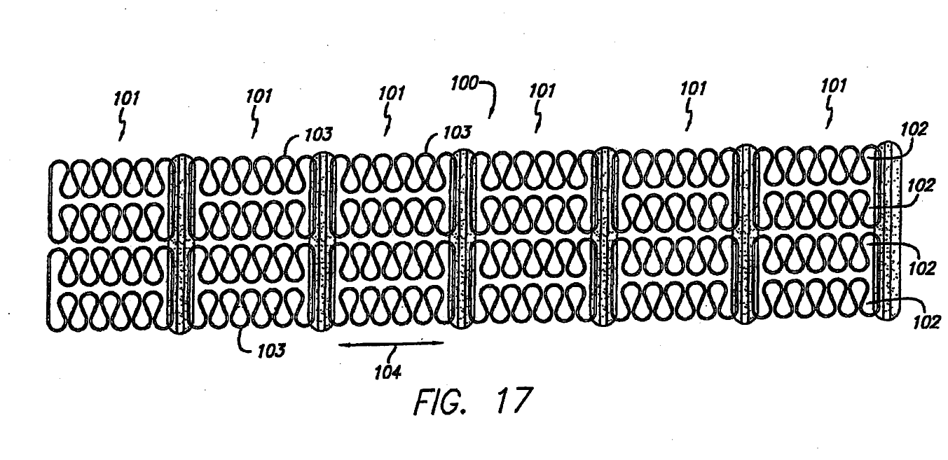

As shown in FIG. 17, cardiac harness 100 includes multiple panels 101 similar to those previously described. Further, undulating strands 102 form the panels and have multiple spring elements 103 that expand and contract along directional line 104, also as previously described. In the cardiac harness 100 shown in FIG. 17, the amplitude of the spring elements is relatively smaller than in other embodiments, and the pitch is higher, meaning there are more spring elements per unit of length relative to other embodiments. Thus, the cardiac harness 100 should generate higher bending forces as the heart expands and contracts during the diastolic and systolic cycles. In other words, the spring elements 103 of cardiac harness 100 will resist expansion, thereby imparting higher compressive forces on the wall of the heart during the diastolic function and will release these higher bending forces during the systolic function as the heart contracts. It may be important to provide undulating strands 102 that alternate in amplitude and pitch within a panel, starting at the base of the harness and extending toward the apex. For example, the pitch and amplitude of an undulating strand closer to the base or the harness may be configured to impart higher compressive forces on the epicardial surface of the heart than the undulating strands closer to the apex or the lower part of the harness. It also may be desirable to alternate the amplitude and pitch of the spring elements from one undulating strand to the next. Further, where multiple panels are provided, it may be advantageous to provide one amplitude and pitch of the spring elements of the undulating strands of one panel, and a different amplitude and pitch of the spring elements of the undulating strands of an adjacent panel. The FIG. 17 embodiment can be configured with electrodes as previously described in other embodiments, or with coils, both of which assist with the delivery of the cardiac harness by providing column support to the harness.

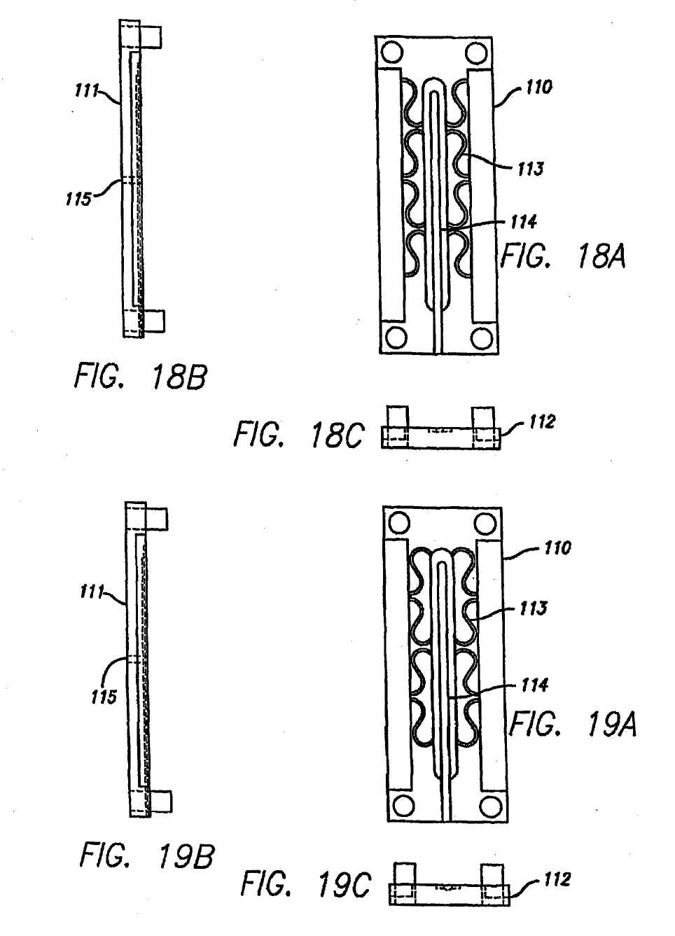

The cardiac harness, having either electrodes or coils, can be formed using injection molding techniques as shown in FIGS. 18A-18C and 19A-19C. The molds in FIGS. 18A-18C are substantially the same as the molds shown in FIGS. 19A-19C, with the exception of the undulating pattern grooves that receive the undulating strands previously described. In referring to FIG. 18A, bottom mold 110 includes a pattern for receiving the cardiac harness and a coil or an electrode. For illustration purposes, FIG. 18B shows top mold 111 and FIG. 18C shows end view mold 112. The top mold mates with the bottom mold. As can be seen, the cardiac harness undulating strands will fit in undulating strand groove 113, which extend into coil groove 114. The previously described electrodes or coils fit into coil grooves 114. Injection port 115 is positioned midway along the mold fixtures, however, more than one injection port can be used to insure that the flow of polymer is uniform and consistent. Preferably, silicone rubber is injected into the molds so that the silicone rubber flows over the undulating strands and the electrodes or the coils. When the cardiac harness assembly is taken out of the mold, the undulating strands will be attached to the electrodes or the coils by the silicone rubber according to the pattern shown. Other patterns may be desired and the molds are easily altered to provide any pattern that ensures a secure attachment between the undulating strands and the electrodes or the coils. Importantly, the molds of FIGS. 18 and 19 can be used to inject the dielectric material or silicone rubber inside the coils and, if necessary, between the gaps in the coils in order to insure that the coils and the undulating strands are insulated from each other. The silicone rubber fills the inside of the coils, extrudes through the gaps in the coils, and forms a skin on the inner and outer surface of the coil. This skin is selectively removed (as will be described) to expose portions of the electrode coils so that they can conduct current as described. Further, the coils and the undulating strands do not overlap or touch in order to reduce any frictional engagement between the metallic coils and the metallic undulating strands. In order to increase the frictional engagement between the cardiac harness and the epicardial surface of the heart, small projections (not shown) can be molded along the surface of the coils that will contact the epicardial surface. As previously described with respect to the grip pads, these small projections, preferably formed of silicone rubber, will engage the epicardial surface of the heart and increase the frictional engagement between the coils and the surface of the heart in order to secure the harness to the heart without the use of sutures, clips, or other mechanical attachment means.

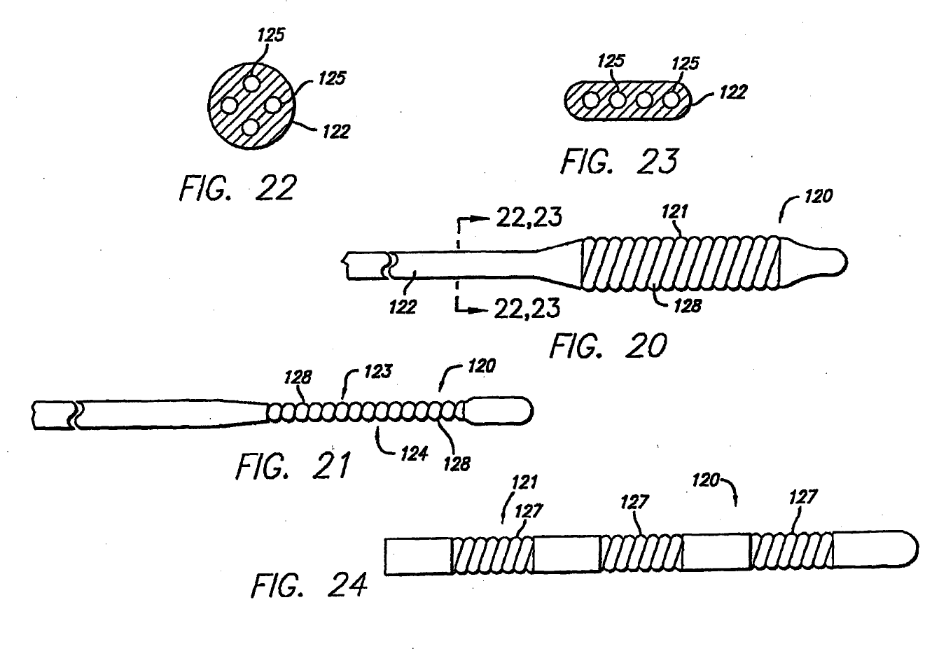

As shown in FIGS. 20-23, a portion of a lead having an electrode 120 is shown in the form of a conductive coil 121. The coil can be formed of any suitable wire that is conductive so that an electrical shock can be transmitted through the electrode and through the myocardium of the heart. In this embodiment, the coil wire is wrapped around a dielectric material 122 in a helical configuration, however, a spiral wrap or other configuration is possible as long as the coil has superior fatigue resistance and longitudinal flexibility. Importantly, conductive coils 121 have high fatigue resistance which is necessary since the coil is on or near the surface of the beating heart so that the coil is constantly flexing along its longitudinal length in response to heart expansion and contraction. The cross-section of the wire preferably is round or circular, however, it also can be oval shaped or flat (rectangular) in order to reduce the profile of the electrode for minimally invasive delivery. A circular, oval or flat wire will have a relatively high fatigue resistance as well as a relatively low profile for delivery purposes. Also, a flat wire coil is highly flexible along the longitudinal axis and it has a relatively high surface area for delivering an electrical shock. The electrode 120 has a first surface 123 and a second surface 124. The first surface 123 will be proximate the epicardial surface of the heart, or other portions of the heart, while the second surface will be opposite the first surface and away from the epicardial surface of the heart. A conductive wire (not shown) extends through the dielectric material 122 and attaches to the coil wire 121 at one or more locations along the coil or coils, and the conductive wire is connected to a power source (e.g., an ICD) at its other end. As shown in FIG. 22, the cross-section of the electrode 120 can be circular, or as shown in FIG. 23, can be oval for reduced profile for minimally invasive delivery. Other cross-sectional shapes for electrode 120 are available depending upon the particular need. All of these cross-sectional shapes will have relatively high fatigue resistance. As shown in FIGS. 22 and 23, multiple lumens 125 can be provided to carry one or more conductive wires from the electrode to the power source (pulse generator, ICD, CRT-D, pacemaker, etc.). The lumens also can carry sensing wires that transmit data from a sensor on or in the heart to a pacemaker so that the heart can be monitored. Further, the lumens 125 can be used for other purposes such as drug delivery (therapeutic drugs, steroids, etc.), dye injection for visibility under fluoroscopy, carrying a guide wire (not shown) or a stylet to facilitate delivery of the electrodes and the harness, or for other purposes. The lumens 125 can be used to carry a guide wire (not shown) or a stylet in such a way that the column stiffness of the coil is increased by the guide wire or stylet, or in a manner that will vary the column stiffness as required. By varying the column stiffness of the coils with a guide wire or a stylet in lumens 125, the ability to push the cardiac harness over the heart (as will be described) will be enhanced. The guide wires or stylets also can be used, to some extent, to steer the coils and hence the cardiac harness during delivery and implantation over the heart. The guide wire or stylet in lumens 125 can be removed after the cardiac harness is implanted so that the coils (electrodes) become more flexible and atraumatic.

As shown in FIGS. 20-23, the electrode 120 not only provides an electrical conduit for use in defibrillation, but also has sufficient column strength when attached to the cardiac harness to assist in the delivery of the harness by minimally invasive means. As will be further described, the coils 121 provide a highly flexible electrode along its longitudinal length, and also provide a substantial amount of column strength when coupled with a cardiac harness to assist in the delivery of the harness.

As further shown in FIGS. 20-23, a dielectric material such as silicone rubber 126 can be used to coat electrodes 120. During the molding process (previously described), when the electrode 120 is attached to the cardiac harness, silicone rubber 126 will coat the entire electrode 120. Soda blasting (or other known material removal process) can be used to remove portions of the silicone rubber skin from the coils 121 in order to expose first surface 123 and second surface 124 (or portions of those surfaces) so that the bare metal coil is exposed to the epicardial surface of the heart. Preferably, the silicone rubber is removed from both the first surface and the second surface, however, it also may be advantageous to remove the silicone rubber from only the first surface, which is proximate to or in contact with the epicardial surface of the heart. The electrode 120 has a surface area 128 which essentially includes all of the bare metal surface area that is exposed and that will deliver a shock. The amount of surface area per electrode can vary greatly depending upon a particular application, however, surface areas in the range from about 50 mm² to about 600 mm² are typical. While it is possible to remove the silicone rubber from only the second surface (facing away from the heart), and leaving the first surface coated with silicone rubber, an electrical shock can still be delivered from the bare metal second surface, however, the electrical shock delivered may not be as efficient as with other embodiments. While the dimensions of the electrodes can vary widely due to the variations in the size of the heart to be treated in conjunction with the size of the cardiac harness, generally the length of the electrode ranges from about 2 cm to about 16 cm. The coil 121 has a length in the range of about 1 cm to about 12 cm. Commercially available leads having one or more electrodes are available from several sources and may be used with the cardiac harness of the present invention. Commercially available leads with one or more electrodes is available from Guidant Corporation (St. Paul, Minn.), St. Jude Medical (Minneapolis, Minn.) and Medtronic Corporation (Minneapolis, Minn.). Further examples of commercially available cardiac rhythm management devices, including defibrillation and pacing systems available for use in combination with the cardiac harness of the present invention (possibly with some modification) include, the CONTAK CD®, the INSIGNIA® Plus pacemaker and FLEXTREND® leads, and the VITALITY™ AVT® ICD and ENDOTAK RELIANCE® defibrillation leads, all available from Guidant Corporation (St. Paul, Minn.), and the InSync System available from Medtronic Corporation (Minneapolis, Minn.).

As shown in FIG. 24, the conductive coils 121 need not be continuous along the length of the electrode 120, but can be spatially isolated or staggered along the electrode. For example, multiple coil sections 127, similar to the coil 121 shown in FIG. 20, can be spaced along the electrode with each coil section being attached to the conductive wire so it receives electrical current from the power source. The coil sections can be from about 0.5 cm to about 2.0 cm long and be spaced from about 0.5 cm to about 4 cm apart along the electrode. The dimensions used herein are by way of example only and can vary to suit a particular application

When removing portions of the silicone rubber from the electrode 120 using soda blasting or a similar technique, it may be desirable to leave portions of the electrode masked or insulated so that the masked portion is non-conductive. By masking portions of two electrodes positioned, for example, on opposite sides of the left ventricle, it is possible to vector a shock at a desirable angle through the myocardium and ventricle. The shock will travel from the bare metal (unmasked) portion of one electrode through the myocardium and the ventricle to the bare metal (unmasked) portion of the opposing electrode at a vector angle determined by the position of the masking on the electrodes.

The associated cardiac rhythm management devices are implantable devices that provide electrical stimulation to selected chambers of the heart in order to treat disorders of cardiac rhythm and can include pacemakers and implantable cardioverter/defibrillators and/or cardiac resynchronization therapy devices (CRT-D). A pacemaker is a cardiac rhythm management device which paces the heart with timed pacing pulses. As previously described, common conditions for which pacemakers are used is in the treatment of bradycardia (ventricular rate is too slow) and tachycardia (cardiac rhythms are too fast). As used herein, a pacemaker is any cardiac rhythm management device with a pacing functionality, regardless of any other functions it may perform such as the delivery of cardioversion or defibrillation shocks to terminate atrial or ventricular fibrillation. An important feature is to provide a cardiac harness having the capability of providing a pacing function in order to treat the synchrony of both ventricles. To accomplish the objective, a pacemaker with associated leads and electrodes are associated with and incorporated into the cardiac harness of the present invention. The pacing/sensing electrodes, alone or in combination with defibrillating electrodes, provide treatment to synchronize the ventricles and improve cardiac function.

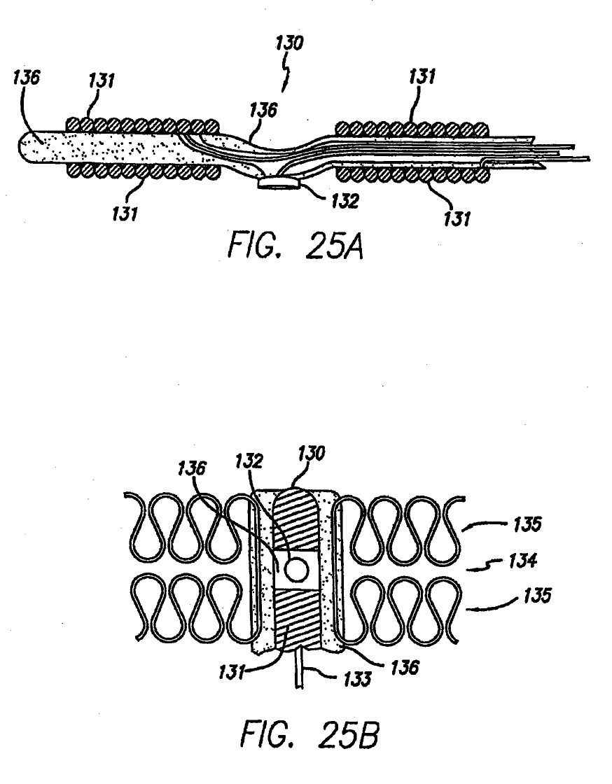

A pacemaker and a pacing/sensing electrode are incorporated into the design of the cardiac harness. As shown in FIGS. 25A and 25B, a lead (not shown) having a defibrillator electrode 130 at its distal end, shown in partial section, not only incorporates wire coils 131 used to deliver a defibrillating electrical shock to the epicardial surface of the heart, but also incorporates a pacing/sensing electrode 132. The defibrillator electrode 130 can be attached to any cardiac harness embodiment previously described herein. In this embodiment, a non-penetrating pacing/sensing electrode 132 is combined with the defibrillating electrode 130 in order to provide data relating to heart function. More specifically, the pacing/sensing electrode 132 does not penetrate the myocardium in this embodiment, however, it may be beneficial in other embodiments for the pacing or sensing electrode to penetrate the myocardium. One advantage of a non-penetrating pacing/sensing electrode is that there is no danger of puncturing a coronary artery or causing further trauma to the epicardium or myocardium. It is also easier to design since there is no requirement of a penetration mechanism (barb or screw) on the pacing/sensing electrode. The pacing/sensing electrode 132 is in direct contact with the epicardial surface of the heart and will provide data via lead wire 133 to the pulse generator (pacemaker), which will interpret the data and provide any pacing function necessary to achieve, for example, ventricular resynchronization therapy, left ventricular pacing, right ventricular pacing, synchrony of both ventricles, and/or biventricular pacing. As shown in FIG. 25B, the pacing/sensing electrode 132 is incorporated into a portion of a cardiac harness 134, and more particularly the undulating strands 135 are attached by dielectric material 136 to the pacing/sensing electrode. As can be seen in FIGS. 25A and 25B, the wire coils 131 of the defibrillating electrode 130 are wrapped around the dielectric material 136, and the dielectric material insulates the pacing/sensing electrode 132 from both the wire coils 131 and from the undulating strands 135 of the cardiac harness. Multiple pacing/sensing electrodes 132 can be incorporated along defibrillating electrode 130, and multiple pacing and sensing electrodes can be incorporated on other electrodes associated with the cardiac harness.

Multi-site pacing (as previously shown in FIGS. 8A-8D) using pacing/sensing electrodes 132 enables resynchronization therapy in order to treat the synchrony of both ventricles. Multi-site pacing allows the positioning of the pacing/sensing electrodes to provide bi-ventricular pacing or right ventricular pacing, left ventricular pacing, depending upon the patient's needs.

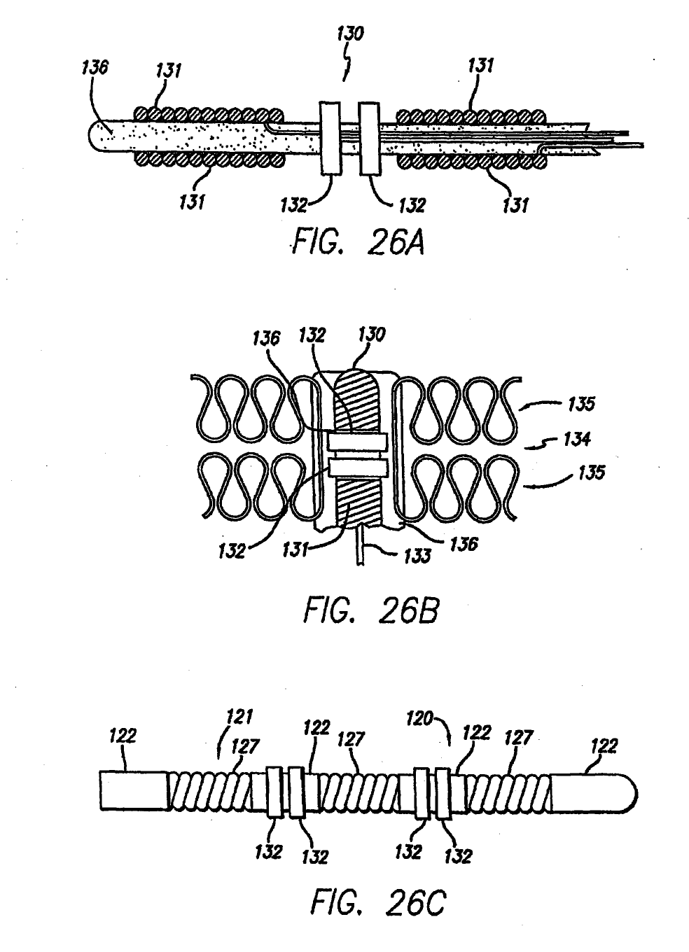

As shown in FIGS. 26A-26C, a defibrillating electrode is combined with pacing/sensing electrodes, for attachment to any of the cardiac harness embodiments disclosed herein. There, the defibrillating electrode 130 is formed of wire coils 131 wrapped in a helical manner. The helical wire can be a wound wire having a single strand or a quadrafilar wire having four wires bundled together to form the coil. The wire coils 131 are wrapped around dielectric material 136 in a manner similar to that described for the embodiments in FIGS. 25A and 25B. The pacing/sensing electrode 132 is in the form of a single ring for unipolar operation, and two rings for bi-polar operation. The pacing/sensing electrode rings 132 are mounted coaxially with the defibrillating electrode wire coils 131, and the conducting wires from the wire coils and the pacing/sensing ring electrode are shown extending through the dielectric material 136 and being insulated from each other. The conducting wires from the defibrillating electrode 130 and from the pacing/sensing ring electrodes 132 can be bundled into a common lead wire 133 which extends to the pulse generator (an ICD, CRT-D, and/or a pacemaker). As can be seen in FIGS. 26A-26C, the pacing/sensing electrode rings 132 have a diameter that is somewhat larger than the defibrillator electrode coils 131 in order to insure preferential contact by the electrode rings against the epicardial surface of the heart. Preferably, several pairs of pacing/sensing electrode rings (bipolar) would be positioned on the cardiac harness and be positioned to come into contact with, for example, the left ventricle free wall. Multi-site pacing allows the pacing/sensing electrode rings 132 to be used for both pacing and resynchronization concurrently. Further, the pacing/sensing electrode rings 132 also can be used in the absence of defibrillating electrodes 130. The prior disclosure relating to molding of the cardiac harness to the defibrillator electrode applies equally as well to the pacing/sensing electrode rings. The wire coil 131 and the pacing/sensing electrode rings 32 can be fabricated in several ways including by laser cutting stainless steel tubing or using highly conductive materials in wire form, such as biocompatible platinum wire. As previously disclosed, the wire coils 131 can be quadrafilar wire (platinum) for improved flexibility and conformability to the epicardial surface of the heart and be biocompatible. The surface of the pacing/sensing electrodes can vary greatly depending upon the application. As an example, in one embodiment, the surface area of the pacing/sensing electrodes are in the range from about 2 mm² to about 12 mm², however, this range can vary substantially. While the disclosed figures show the pacing/sensing electrodes combined with the defibrillating electrodes, the pacing/sensing electrodes can be formed separately and mounted on the cardiac harness with or without defibrillating electrodes.

The defibrillating electrode 130, can be used with commercially available pacing/sensing electrodes and leads. For example, Oscor (Model HT 52PB) endocardial/passive fixation leads can be integrated with the defibrillator electrode 130 by molding the leads into the fibrillator electrode using the same molds previously disclosed herein.

The incorporation of cardiac rhythm management devices into the cardiac harness combines several treatment modalities that are particularly beneficial to patients suffering from congestive heart failure. The cardiac harness provides a compressive force on the heart thereby relieving wall stress, and improving cardiac function. The defibrillating and pacing/sensing electrodes associated with the cardiac harness, along with ICD's and pacemakers, provide numerous treatment options to correct for any number of maladies associated with congestive heart failure. In addition to the defibrillation function previously described, the cardiac rhythm devices can provide electrical pacing stimulation to one or more of the heart chambers to improve the coordination of atrial and/or ventricular contractions, which is referred to as resynchronization therapy. Cardiac resynchronization therapy is pacing stimulation applied to one or more heart chambers, typically the ventricles, in a manner that restores or maintains synchronized bilateral contractions of the atria and/or ventricles thereby improving pumping efficiency. Resynchronization pacing may involve pacing both ventricles in accordance with a synchronized pacing mode. For example, pacing at more than one site (multi-site pacing) at various sites on the epicardial surface of the heart to desynchronize the contraction sequence of a ventricle (or ventricles) may be therapeutic in patients with hypertrophic obstructive cardiomyopathy, where creating asynchronous contractions with multi-site pacing reduces the abnormal hyper-contractile function of the ventricle. Further, resynchronization therapy may be implemented by adding synchronized pacing to the bradycardia pacing mode where paces are delivered to one or more synchronized pacing sites in a defined time relation to one or more sensing and pacing events. An example of synchronized chamber-only pacing is left ventricle only synchronized pacing where the rate in synchronized chambers are the right and left ventricles respectively. Left-ventricle-only pacing may be advantageous where the conduction velocities within the ventricles are such that pacing only the left ventricle results in a more coordinated contraction by the ventricles than by conventional right ventricle pacing or by ventricular pacing. Further, synchronized pacing may be applied to multiple sites of a single chamber, such as the left ventricle, the right ventricle, or both ventricles. The pacemakers are typically implanted subcutaneously on a patient's chest and have leads threaded to the pacing/electrodes as previously described in order to connect the pacemaker to the electrodes for sensing and pacing. The pacemakers sense intrinsic cardiac electrical activity through the electrodes disposed on the surface of the heart. Pacemakers are well known in the art and any commercially available pacemaker or combination defibrillator/pacemaker can be used.

The cardiac harness and the associated cardiac rhythm management device system can be designed to provide left ventricular pacing. In left heart pacing, there is an initial detection of a spontaneous signal, and upon sensing the mechanical contraction of the right and left ventricles. In a heart with normal right heart function, the right mechanical atrio-ventricular delay is monitored to provide the timing between the initial sensing of right atrial activation (known as the P-wave) and right ventricular mechanical contraction. The left heart is controlled to provide pacing which results in left ventricular mechanical contraction in a desired time relation to the right mechanical contraction, e.g., either simultaneous or just preceding the right mechanical contraction. Cardiac output is monitored by impedence measurements and left ventricular pacing is timed to maximize cardiac output. The proper positioning of the pacing/sensing electrodes disclosed herein provides the necessary sensing functions and the resulting pacing therapy associated with left ventricular pacing.

An important feature is the minimally invasive delivery of the cardiac harness and the cardiac rhythm management device system which will be described immediately below.

Delivery of the cardiac harness 20, 60, and 100 and associated electrodes and leads can be accomplished through conventional cardio-thoracic surgical techniques such as through a median sternotomy. In such a procedure, an incision is made in the pericardial sac and the cardiac harness can be advanced over the apex of the heart and along the epicardial surface of the heart simply by pushing it on by hand. The intact pericardium is over the harness and helps to hold it in place. The previously described grip pads and the compressive force of the cardiac harness on the heart provide sufficient attachment means of the cardiac harness to the epicardial surface so that sutures, clips or staples are unnecessary. Other procedures to gain access to the epicardial surface of the heart include making a slit in the pericardium and leaving it open, making a slit and later closing it, or making a small incision in the pericardium.

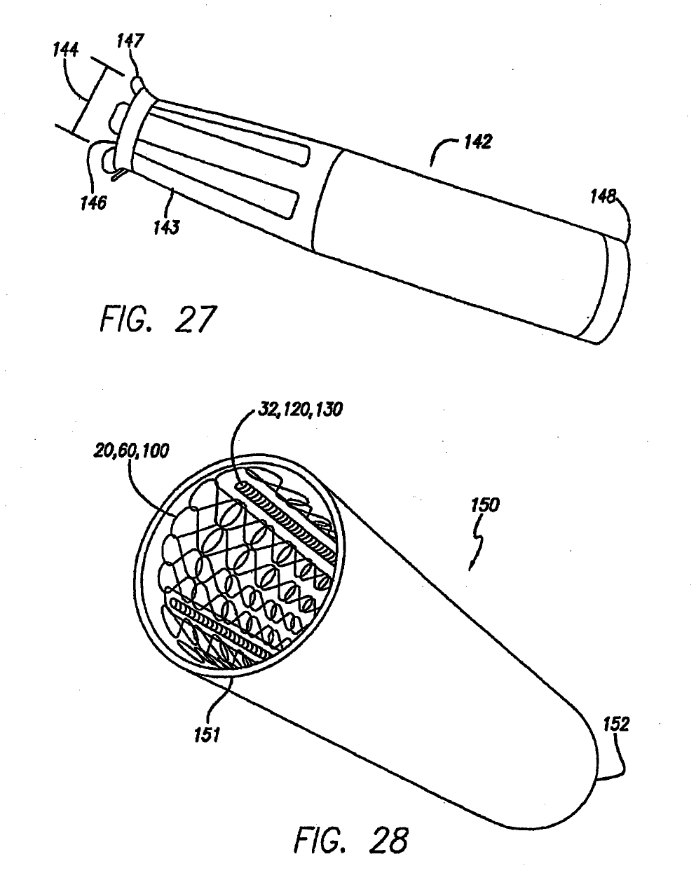

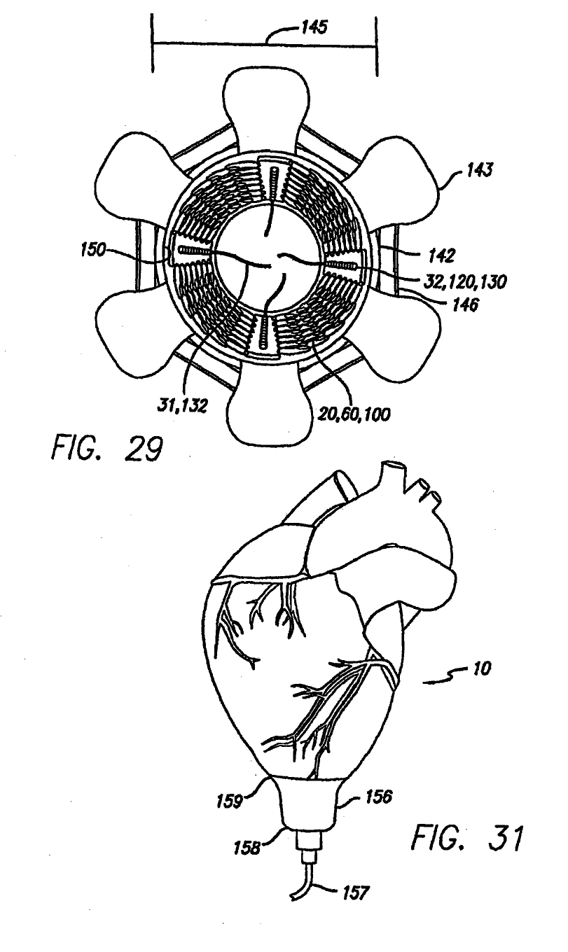

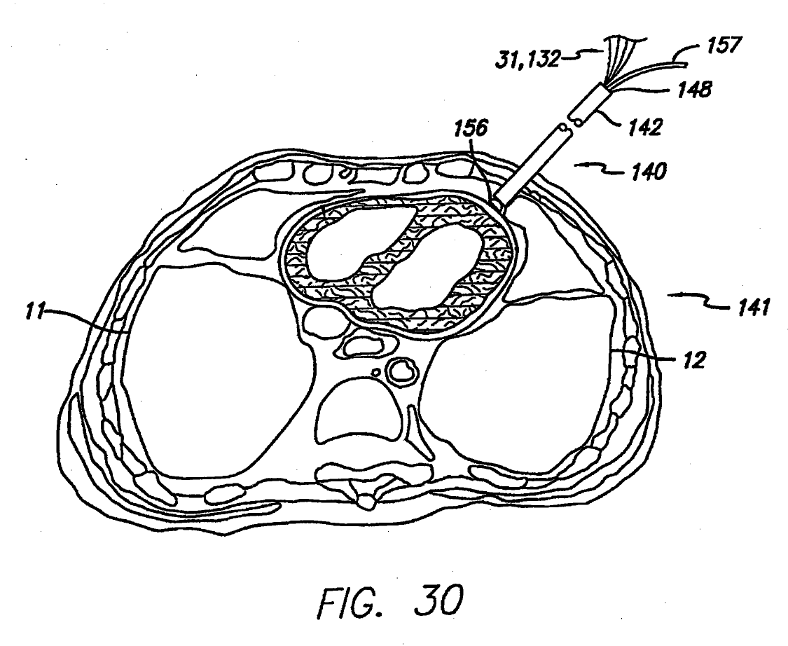

Preferably, however, the cardiac harness and associated electrodes and leads may be delivered through minimally invasive surgical access to the thoracic cavity, as illustrated in FIGS. 27-36, and more specifically as shown in FIG. 30. A delivery device 140 may be delivered into the thoracic cavity 141 between the patient's ribs to gain direct access to the heart 10. Preferably, such a minimally invasive procedure is accomplished on a beating heart, without the use of cardio-pulmonary bypass. Access to the heart can be created with conventional surgical approaches. For example, the pericardium may be opened completely or a small incision can be made in the pericardium (pericardiotomy) to allow the delivery system 140 access to the heart. The delivery system of the disclosed embodiments comprises several components as shown in FIGS. 27-36. As shown in FIG. 27, an introducer tube 142 is configured for low profile access through a patient's ribs. A number of fingers 143 are flexible and have a delivery diameter 144 as shown in FIG. 27, and an expanded diameter 145 as shown in FIG. 29. The delivery diameter is smaller than the expanded diameter. An elastic band 146 expands around the distal end 147 of the fingers and prevents the fingers from overexpanding during delivery of the cardiac harness. The distal end of the fingers is the part of the delivery device 140 that is inserted through the patient's ribs to gain direct access to the heart.

The delivery device 140 also includes a dilator tube 150 that has a distal end 151 and a proximal end 152. The cardiac harness 20, 60, 100 is collapsed to a low profile configuration and inserted into the distal end of the dilator tube, as shown in FIG. 28. The dilator tube has an outside diameter that is slightly smaller than the inside diameter of the introducer tube 142. As will be discussed more fully herein, the distal end 151 of the dilator tube is inserted into the proximal end 147 of the introducer tube in close sliding engagement and in a slight frictional engagement. The slidable engagement between the dilator tube and the introducer tube should be with some mild resistance, however, there should be unrestricted slidable movement between the two tubes. The distal end 151 of the dilator tube will expand the fingers 143 of the introducer tube 142 as the dilator tube is pushed distally into the introducer tube as shown in FIG. 29. In the embodiments shown in FIGS. 27 36, the cardiac harness 20, 60, 100 is equipped with leads (previously described) having electrodes for use in defibrillation or pacing functions.

As shown in FIG. 31, the delivery system 140 also includes a releasable suction device, such as suction cup 156 at the distal end of the delivery device. The negative pressure suction cup 156 is used to hold the apex of the heart 10. Negative pressure can be applied to the suction cup using a syringe or other vacuum device commonly known in the art. A negative pressure lock can be achieved by a one-way valve stop-cock or a tubing clamp, also known in the art. The suction cup 156 is formed of a biocompatible material and is preferably stiff enough to prevent any negative pressure loss through the heart while manipulating the heart and sliding the cardiac harness 20, 60, 100 onto the heart. Further, the suction cup 156 can be used to lift and maneuver the heart 10 to facilitate advancement of the harness or to allow visualization and surgical manipulation of the posterior side of the heart. The suction cup has enough negative pressure to allow a slight pulling in the proximal direction away from the apex of the heart to somewhat elongate the heart (e.g., into a bullet shape) during delivery to facilitate advancing the cardiac harness over the apex and onto the base portion of the heart. After the suction cup 156 is attached to the apex of the heart and a negative pressure is drawn, the cardiac harness, which has been releasably mounted in the distal end 151 of the dilator tube 150, can be advanced distally over the heart, as will be described more fully herein.

As shown in FIG. 30, the delivery device 140, and more specifically introducer tube 142, has been advanced through the intercostal space between the patient's ribs during insertion of the introducer tube, the fingers 143 are in their delivery diameter 144, which is a low profile for ease of access through the small port made through the patient's ribs. Thereafter, the dilator tube 150, with the cardiac harness 20, 60, 100 mounted therein, is advanced distally through the introducer tube so that the fingers 143 are expanded until they achieve their expanded diameter 145. The suction cup 156 can be attached to the apex 13 of the heart 10 either before or after the dilator tube is advanced to spread the fingers 143 of the introducer tube 142. Preferably, the dilator tube has already expanded the fingers on the introducer tube so that there is a larger opening for the suction cup as it is advanced through the inside of a dilator tube, out of the distal end of the introducer tube, and placed in contact with the apex of the heart. Thereafter, a negative pressure is drawn allowing the suction cup to securely attach to the apex of the heart. Visualizing equipment that is commonly known in the art may be used to assist in positioning the suction cup to the apex. For example, fluoroscopy, magnetic resonance imaging (MRI), dye injection to enhance fluoroscopy, and echocardiography, and intracardiac, transesophageal, or transthoracic echo, all can be used to enhance positioning and in attaching the suction cup to the apex of the heart. After negative pressure is drawn and the suction cup is securely attached (releasably) to the apex of the heart, the heart can then be maneuvered somewhat by pulling on the tubing 157 attached to the suction cup, or by manipulating the introducer tube 142, the dilator tube 150, both in conjunction with the suction cup. As previously described, it may be advantageous to pull on the tubing 157 to allow the suction cup to pull on the apex of the heart and elongate the heart somewhat in order to facilitate sliding the harness over the epicardium.

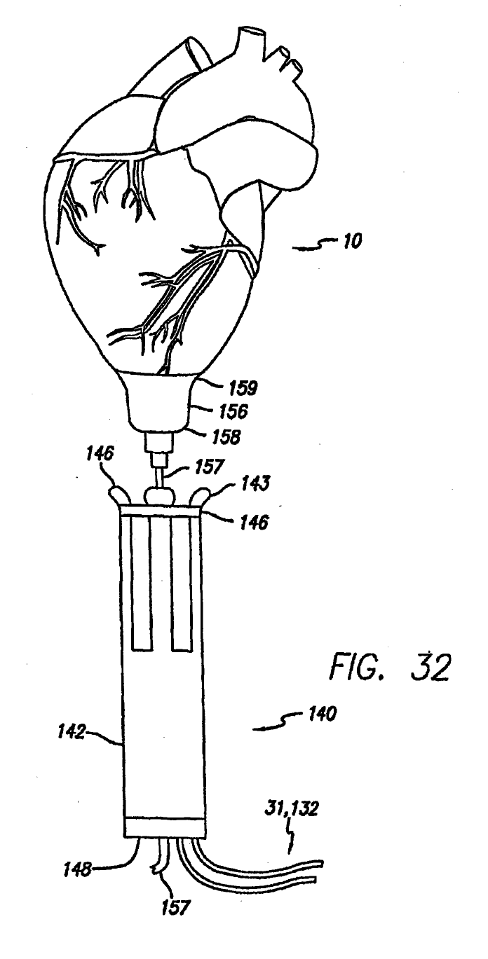

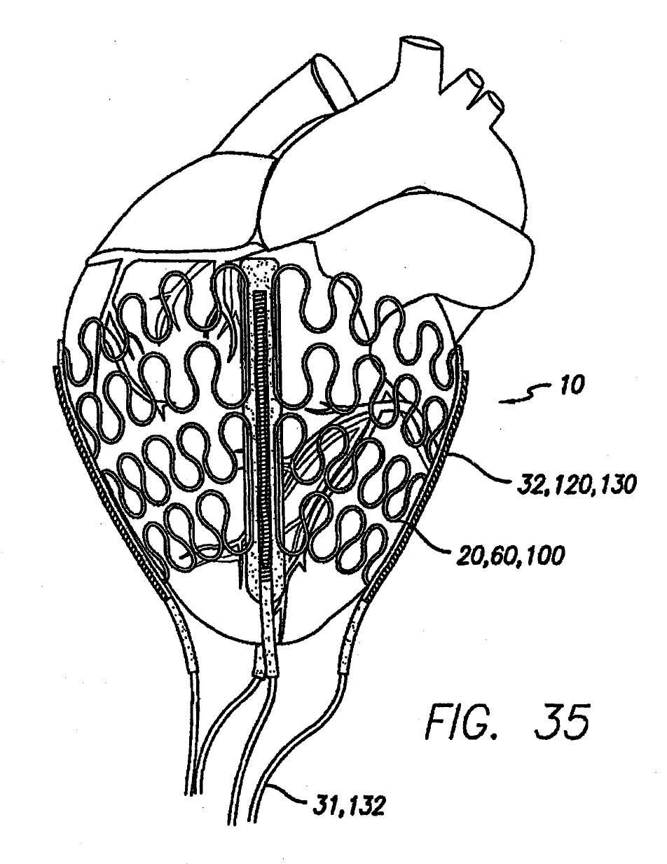

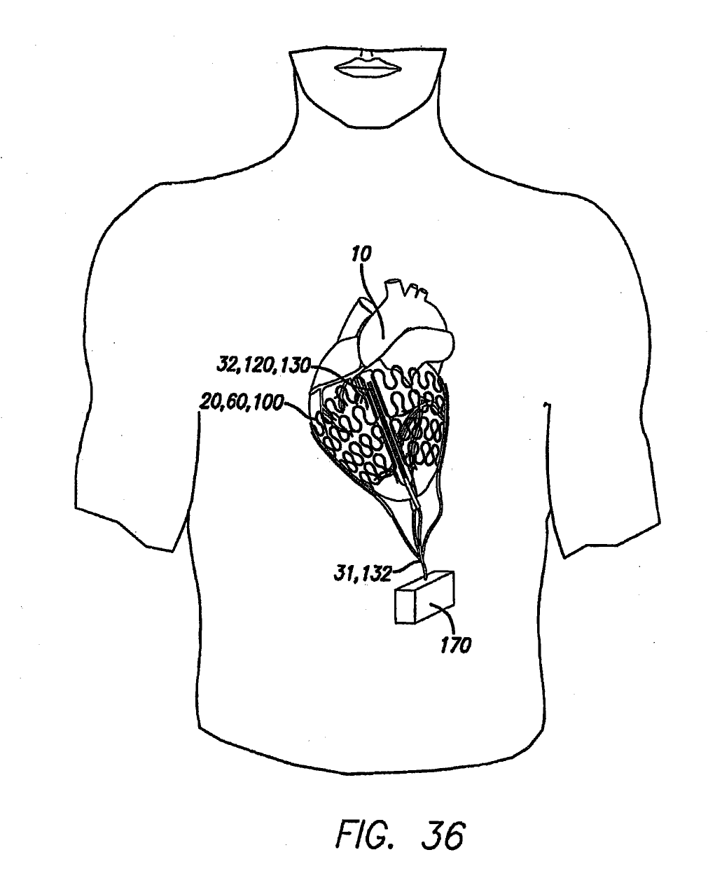

As more clearly shown in FIGS. 32-36, the cardiac harness 20, 60, 100 is advanced distally out of the dilator tube and over the suction cup 156. The suction cup is tapered so that the distal end of the harness slides over the narrow portion of the taper (the proximal end of the suction cup 158). The suction cup becomes wider at its distal end where it is attached to the apex of the heart, and the cardiac harness continues to slide and expand over the suction cup as it is advanced distally. As the cardiac harness continues to be advanced distally, it slides over the apex of the heart and continues to expand as it is pushed out of the dilator tube and along the epicardial surface of the heart. Since the harness and the electrodes 32, 120, 130 are coated with the previously described dielectric material, preferably silicone rubber, the cardiac harness should slide easily over the epicardial surface of the heart. The silicone rubber offers little resistance and the epicardial surface of the heart has sufficient fluid to allow the harness to easily slide over the wet surface of the heart. The pericardium previously has been cut so that the cardiac harness is sliding over the epicardial surface of the heart with the pericardium over the cardiac harness to help hold it onto the surface of the heart. As shown in FIGS. 35 and 36, the cardiac harness 20, 60, 100 has been completely advanced out of the dilator tube so that the harness covers at least a portion of the heart 10. The suction cup 156 has been withdrawn, and the introducer tube 142 and dilator tube 150 also have been withdrawn proximally from the patient. Prior to removing the introducer tube, a power source 170 (such as an ICD, CRT-D, and/or pacemaker) can be implanted by conventional means. The electrodes will be attached to the pulse generator to provide a defibrillating shock or pacing functions as previously described.

As shown in FIGS. 27-36, the cardiac harness 20, 60, 100 was advanced through the dilator tube by pushing on the proximal end of the electrodes 32,120,130, on the lead wires 31, 133, and on the proximal end (apex 26) of the cardiac harness. Even though the electrodes are designed to be atraumatic and longitudinally flexible, the electrodes have sufficient column strength so that pushing on the proximal ends of the electrodes assists in pushing the cardiac harness out of the dilator tube and over the epicardial surface of the heart. The advancement of the cardiac harness may be accomplished by hand, by the physician simply pushing on the electrodes and the leads to advance the cardiac harness out of the dilator tube to slide onto the epicardial surface of the heart.

As shown in the embodiments of FIGS. 27-36, the delivery device 140, and more specifically introducer tube 142 and dilator tube 150, have a circular cross-section. It may be preferable, however, to chose other cross-sectional shapes, such as an oval cross-sectional shape for the delivery device. An oval delivery device may be more easily inserted through the intercostal space between the patient's ribs for a low profile delivery. Further, as the cardiac harness 20, 60, 100 is advanced out of a delivery device 140 having an oval cross-section, the harness distal end will quickly form into a more circular shape in order to assume the configuration of the epicardial surface of the heart as it is advanced distally over the heart.

As shown in FIGS. 35 and 36, the cardiac harness 20, 60, 100 remains firmly attached to the epicardial surface of the heart without the need for any further attachment means, such as sutures, clips, adhesives, or staples. Further, the pericardial sac helps to enclose the harness to prevent it from shifting or sliding on the epicardial surface of the heart.

Importantly, during delivery of the cardiac harness 20, 60, 100, the harness itself, the electrodes 32,120,130, as well as leads 31 and 132 have sufficient column strength in order for the physician to push from the proximal end of the harness to advance it distally through the dilator tube 150. While the entire cardiac harness assembly is flexible, there is sufficient column strength, especially in the electrodes, to easily slide the cardiac harness over the epicardial surface of the heart in the manner described.

If the cardiac harness 20, 60, 100 includes coils 72, as opposed to the electrodes and leads, the harness can be delivered in the same manner as previously described with respect to FIGS. 27-36. The coils have sufficient column strength to permit the physician to push on the proximal end of the coils to advance the cardiac harness distally to slide over the apex of the heart and onto the epicardial surface.

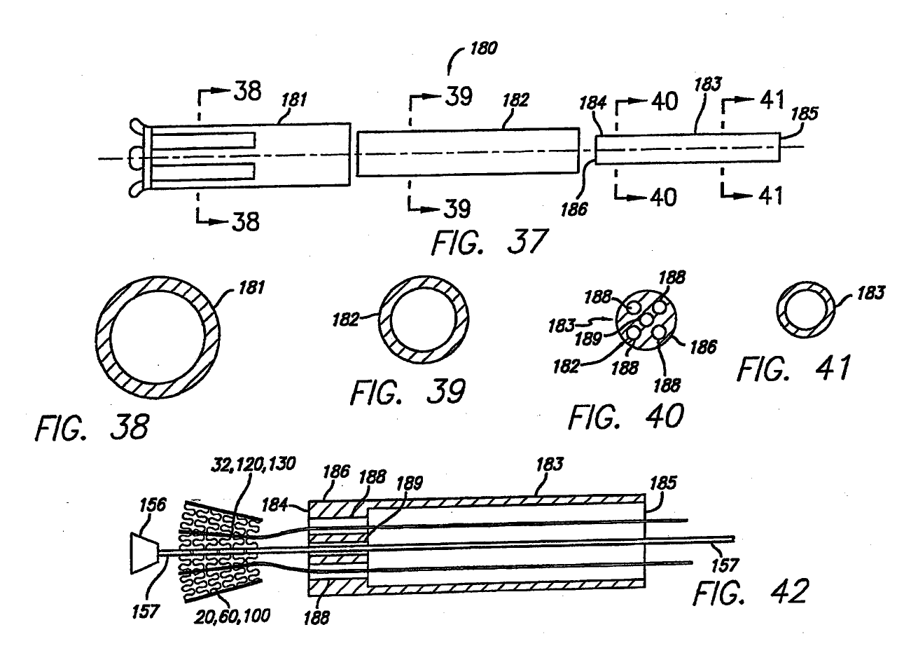

Delivery of the cardiac harness 20, 60, 100 can be by mechanical means as opposed to the hand delivery previously described. As shown in FIGS. 37-42, delivery system 180 includes an introducer tube 181 that functions the same as introducer tube 142. Also, a dilator tube 182, which is sized for slidable movement within the introducer tube, also functions the same as the previously described dilator tube 150. An ejection tube 183 is sized for slidable movement within the dilator tube, that is, the outer diameter of the ejection tube is slightly smaller than the inner diameter of the dilator tube. As shown in FIGS. 40 and 41, the ejection tube has a distal end 184 and a proximal end 185, wherein the distal end of the ejection tube has a plate that fills the entire inner diameter of the ejection tube. The plate has a number of lumens 187 for receiving leads 31, 132 and for receiving the suction cup 156 and associated tubing 157. Thus, lumens 188 are sized for receiving leads 31, 132 therethrough, while lumen 189 is sized for receiving suction cup 156 and the associated tubing 157. The number of lumens 188 in plate 186 will be defined by the number of leads 31,132 associated with the cardiac harness 20, 60, 100. Thus, as shown in FIG. 40, there are four lumens 188 for receiving four leads therethrough, and one lumen 189 for receiving the suction cup 156 and tubing 157 therethrough. The leads and the tubing 157 extend proximally out the proximal end 185 of the ejection tube. As shown in FIG. 42, the suction cup and cardiac harness are on the left side of the schematic, and the ejection tube 183 is on the right hand side of the schematic. For clarity, the dilator tube and the introducer tube have been omitted, however, in practice the cardiac harness would be mounted in the dilator tube, and the dilator tube would extend into the introducer tube, while the ejection tube would extend into the dilator tube. As can be seen in FIG. 42, the leads 31, 132 extend through lumens 188, while the tubing 157 associated with the suction cup extends through lumen 189. The tubing and the leads extend proximally out of the proximal end of the ejection tube, and extend out of the patient during delivery of the harness. As previously described, after the introducer is positioned through the rib cage, and the apex of the heart is acquired by the suction cup, the harness can be advanced out of the dilator by advancing the ejection tube 183 in a distal direction toward the apex of the heart. The leads, the cardiac harness and electrodes all provide sufficient column strength to allow the plate 186 to impart a pushing force against the cardiac harness to advance it distally over the heart as previously described. After the cardiac harness is pushed over the epicardial surface of the heart, the ejection tube can be withdrawn proximally so that the tubing 157 and the leads 31, 132 slide through lumens 189, 188 respectively. The ejection tube 183 continues to be withdrawn proximally so that the proximal end of the leads and the proximal end of tubing 157 are pulled through the distal end 184 of the ejection tube so that the ejection tube is clear of the leads and the tubing.

Suitable materials for the delivery system 140, 180 can include the class of polymers typically used and approved for biocompatible use within the body. Preferably, the tubing associated with delivery systems 140 and 180 are rigid, however, they can be formed of a more flexible material. Further, the delivery systems 140, 180 can be curved rather than straight, or can have a flexible joint in order to more appropriately maneuver the cardiac harness 20, 60, 100 over the epicardial surface of the heart during delivery. Further, the tubing associated with delivery systems 140, 180 can be coated with a lubricious material to facilitate relative movement between the tubes. Lubricious materials commonly known in the art such as Teflon™ can be used to enhance slidable movement between the tubes.

Delivery and implantation of an ICD, CRT-D, pacemaker, leads, and any other device associated with the cardiac rhythm management devices can be performed by means well known in the art. Preferably, the ICD/CRT-D/pacemaker, are delivered through the same minimally invasive access site as the cardiac harness, electrodes, and leads. The leads are then connected to the ICD/CRT-D/pacemaker in a known manner. The ICD or CRT-D or pacemaker (or combination device) may be implanted in a known manner in the abdominal area and then the leads are connected. Since the leads extend from the apical ends of the electrodes (on the cardiac harness) the leads are well positioned to attach to the power source in the abdominal area.

### THE PRESENT INVENTION EMBODIMENTS

Systolic heart failure patients with a depressed left ventricular ejection fraction (usually less than 40%) are the primary targets for cardiac harness therapy. The patients may have either ischemic or non-ischemic etiologies. Patients will have enlarged ventricular dimensions with a reduction in the overall contractility of the heart. A number of beneficial medicaments have already proven to be effective against this disease. Among these are angiotensin-converting-enzyme (ACE) inhibitors, β-blockers, angiotensin II receptor blockers (ARBs), aldosterone antagonists, and diuretics.

One embodiment of the present invention relates to coating a HeartNet™ Implant (which provides ventricular elastic support therapy) with a drug-eluting polymer to enable localized long-term delivery of an anti-hypertrophic/anti-fibrotic therapeutic agent directly to the heart via the pericardial space. The HeartNet™ Implant is currently in clinical trials and is manufactured by Paracor Medical, Inc. (Santa Clara, Calif.). The HeartNet™ Implant is referred to herein as a cardiac harness and is shown, for example in FIG. 5A, except the HeartNet™ Implant does not have electrodes as shown in FIG. 5A. The invention targets the fibrosis and/or hypertrophy associated with systolic heart failure (HF), diastolic HF, and acute myocardial infarction (MI), potentially reversing or limiting these diseases. Furthermore, the invention provides a novel platform that can be used to elute any of a number of pharmacologic agents for the targeted treatment of a range of cardiovascular disorders, thus providing a new paradigm for cardiac therapy: ventricular elastic support therapy (VEST) with local intra-pericardial delivery of a pharmacologic agent.

The cardiac harness having a pharmacologic agent coating provides a combination device which simultaneously provides therapy to the heart via: (1) mechanical support to the failing heart muscle, and (2) localized intra-pericardial delivery of a beneficial pharmacologic agent to reduce hypertrophy and fibrosis. These two features of the novel device are provided by (1) a coated cardiac harness, and by (2) controlled delivery of a suitable pharmacologic agent from a drug-eluting polymer coating applied to the harness. The device will serve to reduce the cardiac hypertrophy and fibrosis that play a role in the pathophysiology of systolic HF, diastolic HF, and HF following acute MI.

mTOR inhibitors are pharmacologic agents that inhibit the mammalian target of rapamycin (mTOR). Rapamycin (sirolimus), and its derivatives including everolimus, zotarolimus, and biolimus A9, act as mTOR inhibitors. Sirolimus has been used in a clinical setting, often in conjunction with calcineurin inhibitors (CNIs), as anti-rejection therapy for organ transplant patients. mTOR inhibitors are also commonly used as anti-proliferative agents on drug-eluting stents (DES). Pre-clinical evidence for their use as cardiac anti-hypertrophic and anti-fibrotic agents has been shown in small-animal models, such as mouse and rat. Preliminary clinical evidence for their utility as cardiac anti-hypertrophic and anti-fibrotic agents exists in transplant patients. Local delivery of mTOR inhibitors to the cardiac tissue has the potential to provide therapeutic levels of anti-hypertrophic and anti-fibrotic mTOR inhibition without the side effects associated with systemic administration.

mTOR inhibitors bind to the cytosolic immunophilin FK Binding Protein-12 (FKBP12) in cells to generate an immunosuppressive complex that, in turn, binds to and inhibits the activation of the mTOR pathway (specifically, mTORC1). mTOR is a key regulatory serine/threonine kinase (phosphotransferase) that regulates cell growth, cell proliferation, cell motility, cell survival, protein synthesis, and transcription. mTOR exerts its effects primarily by switching on and off the cell's translational machinery to affect protein synthesis. Blockade of the mTOR prevents the activation of mTOR targets, thus regulating protein synthesis. Disorders that involve enhanced rates of protein synthesis can lead to tissue hypertrophy (increased cell growth), as in the case of cardiac hypertrophy.

mTOR controls a number of components involved in the initiation and elongation stages of protein synthesis (translation). Each step involves protein factors that are extrinsic to the ribosome, and regulation generally involves alterations in phosphorylation of the protein factors. In a number of cases, the rapid activation of protein synthesis by insulin, growth factors, or other growth-promoting agonists is at least partially inhibited by mTOR inhibitors, implying that mTOR signaling is involved in stimulating the translational machinery.

Systemic dosages of mTOR inhibitors can lead to hypertriglyceridemia, hypercholesterolemia, and vasculitis of the GI tract. Other side effects of systemic mTOR inhibitors include diarrhea, thrombocytopenia, delays in wound healing, rash, hypertension, anemia, hypokalemia, and photosensitivity. The incidence of side effects from systemic dosages of mTOR inhibitors was shown in the ORBIT (Oral Rapamune to Inhibit Restenosis) trial, where 43% of patients receiving 2 mg daily, and 66% of patients receiving 5 mg daily, experienced side effects which ranged from mild to severe in nature. Cessation of oral sirolimus treatment relieved symptoms. These side effects, particularly thrombocytopenia and delays in wound healing, make local delivery of mTOR inhibitors a preferable administration route, particularly for treating HF, where systemic administration is not required.

Drug-Eluting Stents (DES) currently utilize mTOR inhibitors to prevent the re-blockage of arteries post-intervention via local delivery of the mTOR inhibitor to the vessel. The CYPHER™ sirolimus-eluting stent (manufactured by Johnson & Johnson) was approved by the FDA for sale in the United States in April of 2003. The Endeavor DES (manufactured by Boston Scientific) uses the sirolimus analogue zotarolimus, and has been approved for sale in Europe since April 2005 and in the United States since February 2008. The XIENCE/PROMUS DES (manufactured by Abbott Labs) uses yet another mTOR inhibitor (everolimus), and has very rapidly become the leading DES in the United States since its FDA approval in July 2008. There are several other corporations that also market a DES whose pharmacologic agent is a related mTOR inhibitor. The extensive clinical history associated with DES that incorporate mTOR inhibitors into their coating reflects the safety of these devices. Indeed, millions of patients worldwide have been implanted with these permanent devices over the past decade, and it has been demonstrated that local delivery of mTOR inhibitors to the coronary arteries is efficacious in ameliorating the effects of restenosis.

As noted previously, mTOR inhibitors have been shown to have both anti-hypertrophic and anti-fibrotic effects in animal models and humans. McMullen (McMullen, J. R., et al., Inhibition of mTOR Signaling With Rapamycin Regresses Established Cardiac Hypertrophy Induced by Pressure Overload, Circulation, Vol. 109, pp. 3050-3055, 2004) demonstrated that in mice with compensated or decompensated hypertrophy induced by aortic banding, systemic administration of sirolimus regressed increases in heart size by 68% and 41%, respectively. In decompensated mice, significant decreases in left ventricular end-systolic diameter were also observed with treatment, as were improvements in fractional shortening and EF. Significant decreases in LV end diastolic diameter were observed in compensated sirolimus-treated mice. Gao (Gao, X. M., et al., Inhibition of mTOR reduces chronic pressure-overload cardiac hypertrophy and fibrosis, Journal of Hypertension, Vol. 24, pp. 1663-1670, 2006) has demonstrated that the murine aortic banding model produces LV hypertrophy and fibrosis, that hypertrophy and fibrosis are inter-related events, and that treatment with an mTOR inhibitor reduces these effects. Aortic banding was associated with myocyte enlargement, interstitial fibrosis and enhanced expression of collagen I, collagen III, and ANP, while aortic banding caused decreased expression of α-MHC and SERCA2a. Mice with transverse aortic constriction (TAC) developed LV hypertrophy with increased wall thickness and a 20% increase in LV mass index (LVMI) measured after 5 weeks of TAC. After treatment with sirolimus for 4 weeks, LVMI was significantly decreased (35-45%), LV wall thickening index was decreased, cardiomyocyte size was reduced by 46%, and collagen deposition was suppressed by 38%, when compared to control animals with LV hypertrophy. Throughout sirolimus treatment, LV contractile function was preserved. Another effect of mTOR inhibition in these animals was the restoration of α-MHC and SERCA2a expression to normal levels and the reduction of ANP levels. Furthermore, S6 and eIF4E phosphorylation, which was up-regulated in mice with LV hypertrophy when compared to sham-operated mice, was significantly attenuated. The critical component of these findings is that sirolimus treatment can positively affect chronically established cardiac hypertrophy and fibrosis. Shioi (Shioi, T., et al., Rapamycin Attenuates Load-Induced Cardiac Hypertrophy in Mice, Circulation, Vol. 107, pp. 1664-1670, 2003) also demonstrated that sirolimus acts as an anti-hypertrophic agent in a mouse model of aortic banding. Mice with aortic banding had induced LV hypertrophy with increased S6K1 activation and S6 phosphorylation in the heart resulting from the acute pressure overload. Sirolimus completely inhibited the basal activity and the load-induced increase in S6 phosphorylation. In addition, sirolimus suppressed load-induced increases in heart weight/tibial length by 67%, without affecting body weight, lung weight, or liver weight. Increases in myocyte size were also reduced by 57% with sirolimus. These data provide further evidence for the role of mTOR inhibition in decreasing chamber size in load-induced hypertrophy without compromising systolic function.

A study of rats with MI induced by left anterior descending coronary artery ligation also illustrates the anti-hypertrophic effects of mTOR inhibitors. Animals treated with everolimus experienced a significant reduction in LV end-diastolic diameter and a significant increase in EF, compared to untreated MI rats. In the everolimus-treated animals, myocyte size was also reduced by 33%.

Sirolimus has been implicated as an anti-hypertrophic agent in a study of 58 cardiac transplant patients who were switched from a calcineurin inhibitor (CNI) to sirolimus for anti-rejection therapy. These patients were compared to a control group that remained on the CNI anti-rejection therapy. Patients in the sirolimus arm experienced a significant decrease in LV mass. Systolic and diastolic blood pressure was also decreased in sirolimus patients, while no change was observed in CNI patients. In addition, left atrial volume index (LAVI), which was used as a surrogate for ventricular function, was significantly decreased in sirolimus patients. Of note, patients treated with CNIs saw a slight increase in LV mass with concomitant increase in LAVI. Myocardial biopsies performed in both patient groups showed that a protein induced by mTOR inhibition, p27Kip1, was increased in sirolimus patients and unchanged in CNI patients. These data in combination with the aforementioned animal data suggest that the sirolimus acts directly on the myocardium and has an anti-hypertrophic effect.

Sirolimus has also been implicated as an anti-fibrotic agent in a study of 29 patients maintained on a calcineurin inhibitor (CNI) for 3.8±3.4 years before switching to sirolimus for post-cardiac transplantation anti-rejection therapy. These patients were compared to a control group that remained on the CNI. Intravascular ultrasound was used to show that both mean plaque volume and plaque index were significantly increased in the control patients on CNI therapy, but remained the same in sirolimus patients. These data suggest that in addition to having anti-hypertrophic properties, mTOR inhibitors also have anti-fibrotic effects.

The aforementioned data support the premise that a coated cardiac harness used to deliver an mTOR inhibitor directly to the myocardium via local elution into the pericardial space will lead to a reduction in cardiac hypertrophy and fibrosis, thus benefiting HF patients.

Intra-pericardial delivery of pharmacologic agents offers a promising new technique for providing pharmacologic therapy to the cardiac tissues, while avoiding side effects often associated with systemic administration of efficacious doses. The pericardium provides a natural reservoir in which pharmacologic agents can be administered and distributed to the cardiac tissue while minimizing systemic distribution. Because the cardiac harness resides inside the pericardial space, it provides a vehicle upon which the pharmacologic agent can reside for controlled intra-pericardial delivery.

The drug-eluting cardiac harness platform, once established, can be modified to deliver any of a number of pharmacologic agents for the treatment of HF.

The current basic standard of care for systolic heart failure includes ACE inhibitors and β-blockers. The primary side effect of β-blockers is a lower heart rate. Local drug delivery from the pericardium would not be expected to alter this side effect. ACE inhibitors are very well tolerated and most recent trials show the use of this therapy in around 95% of patients. Many other treatments, including endothelin-receptor antagonists, antibodies against tumor necrosis factor a, and ARBs, have not been found to reduce mortality among patients with left ventricular dysfunction and heart failure who are being treated with this standard of care therapy.

All of these therapies have dose effects and all have systemic side effects. Many of these therapies may be more advantageously delivered via the intrapericardial space. To be effective, the therapeutic agent must provide benefits incremental to this standard of care background therapy.

However, aldosterone blockade reduces total mortality and hospitilization due to progressive heart failure and the rate of sudden death from cardiac causes, as well as the rate of hospitalizations for heart failure, among patients with severe heart failure due to systolic left ventricular dysfunction who are being treated with an ACE inhibitor. Aldosterone blockade also prevents ventricular remodeling and collagen formation in patients with left ventricular dysfunction after acute myocardial infarction and affects a number of pathophysiological mechanisms that are thought to be important in the prognosis of patients with acute myocardial infarction.

Furthermore, aldosterone blockade reduces coronary vascular inflammation and the risk of subsequent development of interstitial fibrosis in animal models of myocardial disease. Aldosterone blockade also reduces oxidative stress, improves endothelial dysfunction, attenuates platelet aggregation, decreases activation of matrix metalloproteinases, and improves ventricular remodeling.

The use of aldosterone blockades is primarily limited by its side effects, the most serious being hyperkalemia. A number of patients cannot tolerate being on the drug at all and for those patients prescribed the drug the dose is limited by the systemic side effects. In recent heart failure studies with strong background medicine for the patients, typically less than 40% of the patients use an aldosterone blockade drug.

In the Randomized Aldosterone Evaluation Study (RALES) study, the aldosterone blocker spironolactone significantly reduced the risk of both morbidity and death among the high-risk heart failure patients with a low incidence of serious hyperkalemia. This safety was attributed to previous efforts determining an effective and safe dose of the drug when used in conjunction with an ACE inhibitor. Spironolactone at a dose of 12.5 to 25 mg daily was effective in blocking the aldosterone receptors and decreasing atrial natriuretic peptide concentrations. Serious hyperkalemia occurs most frequently with daily doses of 50 mg or greater.

In a long-term French study of spironolactone use, the blood pressure decrease was greater with doses of 75 to 100 mg (12.4% and 12.2%) than with doses of 25 to 50 mg (5.3 and 8.5%), but no additional decrease was found with doses above 150 mg. The side-effect of gynecomastia, the development of abnormally large mammary glands in males resulting in breast enlargement, was found to be reversible and dose-related; at doses of 50 mg or less the incidence was 8.9%, but 52.2% for doses of 150 mg or higher.

One embodiment of the present invention is directed to a cardiac harness that is combined with beneficial medicaments, particularly aldosterone blockade, and a system for delivery of the medicament wherein the aldosterone blockade is controllably released to a patient's heart at a dose range of 0.1 to 200 mg per day. The combination of the present invention provides for a novel way to extend the efficacy of a cardiac harness and beneficial medicaments for use in the site-specific treatment of cardiac and non-cardiac maladies.

In one embodiment of the invention, as shown in FIGS. 5A and 6B, a cardiac harness 20 directs the local delivery of an mTOR inhibitor or the combination of an ACE inhibitor, a β-blocker, and aldosterone blockade to one or more specific target areas on or around a mammalian heart 10. For example, the mTOR inhibitor can be coated onto a polymer material 37 on the undulating strands 22 of the cardiac harness 20, which is then mounted directly onto the epicardial surface of the heart. Such localized and targeted delivery of the drug can prevent undesirable systemic side effects by eliminating the circulation of the drug in areas of the body other than the target tissue. Alternately, alodosterone blockade either alone or in combination with either or both an ACE inhibitor or a β-blocker, can be coated onto the dielectric material 37 (silicone rubber 126) on the undulating strands 22 of the cardiac harness 20, which is then mounted directly onto the epicardial surface of the heart. The intact pericardium surrounds the cardiac harness and acts as a barrier to help keep the drugs in direct contact with the epicardium as the drug coatings elute from the polymer or dielectric material 37. The pericardium acts as a natural barrier that also serves to minimize systemic absorption. The pericardium also restricts fluid flow to and away from the surface of the heart, providing a low rate of turnover reservoir in which delivery of a medicament can achieve high and prolonged concentrations around the epicardium. It has been experimentally shown that compounds with different molecular weights and size are cleared differently from the pericardial space; the bigger the molecule, the slower the clearance rate. Pharmacokinetic profiles of intrapericardially applied substances are such that desired local drug concentrations can be obtained at lower dosages, whereas systemic concentrations remain low (thus reducing the potential risk of peripheral side effects). Therefore, intrapericardial application of therapeutic agents provides a promising strategy for site-specific treatment of heart or coronary diseases. Preferably, in the case of the mTOR inhibitor, the mTOR inhibitor will elute at a controlled rate over time for a period of up to five years. The period for elution can range, for example, from one day to one hundred eighty days, and more preferably from thirty days to about one year.

An FDA-approved polymer matrix that is a durable coating technology designed for the site-specific delivery of low-molecular weight drugs, such as mTOR inhibitors, has been identified for use with the cardiac harness. The polymer matrix is currently used in a number of implantable applications, including DES and ophthalmic applications. The matrix has a significant clinical history through its application on the first-to-market coronary DES (CYPHER™). It consists of a proprietary blend of poly-butyl methacrylate (PBMA) and polyethylene vinyl acetate (PEVA) polymers. By varying the ratios of the constituent polymers within the coating, both drug delivery rates and mechanical properties can be controlled. An mTOR inhibitor (with an established Drug Master File) with the PEVA/PMBA polymer coating is applied to the cardiac harness for controlled elution into the pericardial space.

For example, one such coating is the a polymer matrix Bravo™ from Surmodics (Eden Prairie, Minn.). It is a blend of poly-butyl methacrylate (PBMA) and polyethylene vinyl acetate (PEVA) with a tunable elution rate capable of maintaining an elution rate up to two years. Other possible coatings include, but are not limited to, ethyl vinyl alcohol and PLA. These coatings can determine the rate of delivery of the selected medicament, and provide a prolonged effect from the medicament. If the agent is soluble in silicone, the therapeutic drug target may be loaded directly into the harness tubing. In one embodiment, the cardiac harness 20 is coated with a dialectric material (e.g., silicone rubber) (see FIG. 5A). The dielectric material is then coated with a parylene (or one of its derivatives) tie layer so that a drug can be coated onto the parylene layer. The parylene layer may vary in thickness from less than one micrometer to about 0.0762 mm. The rate and extent of release of the therapeutic agent from the delivery source are controlled via the characteristics of the matrix or coating or reservoir as well as by the characteristics of the therapeutic agent. In another embodiment, the drug-release from the cardiac harness is delayed. In this embodiment, an outer coating is applied over the mTOR inhibitor layer such that the outer coating slowly degrades after the cardiac harness is implanted over the epicardium. After the outer coating degrades, which can take days or even months, the mTOR inhibitor begins to elute from the harness.

Intrapericardial delivery for local cardiac therapy has been hampered by the difficulty in accessing the pericardial space and the lack of any usable long-term platforms or structures for use in delivery. Adding medically-beneficial medicaments to the cardiac harness is a novel way to overcome these limitations of systemic beneficial medicament use and of intrapericardial delivery.

The epicardial coronaries are exposed to the pericardial space. With stents and optimal medical therapy, there is still a 12% risk of acute coronary syndrome (ACS) within two years. Intrapericardial drug delivery for local cardiac therapy and pancoronary therapy may prevent the issue. Also, intrapericardial delivery may facilitate the treatment of vulnerable plaque throughout the coronary tree without necessarily having to identify the exact vulnerable plaque to be treated.

It has been shown that intrapericardially delivered agents cause measurable effects in the coronary circulation without systemic side effects. Different concentrations of various proteins between blood and pericardial fluid of different factors may help in regulation of coronary tone or even myocardial function, such as fibroblast growth factor-2 and atrial natriuretic factor. Patients with unstable angina have higher pericardial fluid concentrations of basic fibroblast growth factor as compared with patients undergoing surgery for non-coronary causes.

Another promising pharmacologic therapy for intra-pericardial delivery is a class of proteinases that target the ECM. Collagen degradation has been implicated as a cause of LV dilation. Generally, matrix metalloproteinases (MMPs) break down specific elements of the ECM, leading to dilated cardiomyopathy. A balance of MMPs and tissue inhibitors of MMPs (TIMPs) regulates the ECM; alterations in this balance have been documented in animal models of MI and pressure-overload hypertrophy, and in humans with pressure-overload hypertrophy. A broad-spectrum MMP inhibitor improved ECM composition and LV function, and a selective MMP inhibitor reduced LV dilation and increased EF in animal models of MI. However, “frozen-joint syndrome” was observed in 30% of patients treated systemically with a broad-spectrum MMP inhibitor, and plasma concentration of a selective-spectrum MMP inhibitor was likely too low to show significant results. As such, systemic administration of MMP inhibitors has not been shown as a viable therapeutic option for HF patients. Local delivery may be the only viable option for MMP inhibition as a HF treatment.

The drug-eluting cardiac harness can be readily adopted to incorporate the pharmacologic agents described above, as well as other peptides and pharmacologic agents for future study in the HF population. Alternatively, these agents can be used in conjunction with, or as an alternative to, mTOR inhibitors. In summary, the drug-eluting cardiac harness can be safely placed in the pericardial space of a HF patient using an established surgical procedure. It can be coated with a drug-eluting polymer for controlled intra-pericardial elution of a pharmacologic agent to directly target the cardiac tissue, and may represent an effective platform for targeted intra-pericardial delivery of a broad array of pharmacologic agents.

Paclitaxel delivered intrapericardially has been shown to have the ability to inhibit neointimal proliferation in the coronaries of pigs in response to balloon injury, suggesting that intrapericardially delivered drugs may be used to modulate the inflammatory response in coronaries. Intrapericardially delivered agents thus can prevent restenosis, decrease rethrombosis, unstable angina, myocardial infarction, stabilize vulnerable plaque, and reduce risk of sudden death without the systemic side effects.

In another embodiment shown in FIGS. 20-23, a dielectric material 37 such as silicone rubber 126 can be used to coat electrodes 120. During the molding process (previously described), when the electrode 120 is attached to the cardiac harness 20, silicone rubber 126 will coat at least a portion of the electrode 120. In this embodiment, the silicone rubber 126 of the cardiac harness 20 is coated with a non-biodegradable aldosterone blockade coating. As the aldosterone blockade coating elutes from the silicone rubber jacket 126, it is in direct contact with the epicardium.

A durable drug delivery coating may be applied over the existing harness. For example, one such coating is the a polymer matrix Bravo™ from Surmodics. It is a blend of poly-butyl methacrylate (PBMA) and polyethylene vinyl acetate (PEVA) with a tunable elution rate capable of maintaining an elution rate up to 2 years. Other possible coatings include but are not limited to ethyl vinyl alcohol and PLA. These coatings can determine the rate of delivery of the selected medicament, and provide a prolonged effect from the medicament. If the agent is soluble in silicone, the therapeutic drug target may be loaded directly into the harness tubing. The rate and extent of release of the therapeutic agent from the delivery source are controlled via the characteristics of the matrix or coating or reservoir as well as by the characteristics of the therapeutic agent.

U.S. Pat. No. 7,056,533 (Chudzik et al.), provides for a crosslinkable coating composition for use in delivering a medicament from the surface of a medical device positioned in a patient. Specifically, once crosslinked, the coating composition provides a gel matrix that is adapted to contain the medicament to be released from the matrix in a controlled manner. The Chudzik et al. patent is incorporated herein by reference thereto.

Many factors ultimately determine the dose rate and duration from the coating material: the size and shape, the material type and molecular weight of the matrix material; solubility, biodegradability, and/or hydrophilicity of the coating; permeability factors involving the therapeutic agent and the particular matrix material; degradation of the matrix; and the concentration and kinds of other additives. Porosity in the coating can impact the ease of movement of therapeutic agents from the coating into adjacent cardiac tissue. Coating composition and chemical structure of the therapeutic agent or agents can influence the nature of interaction between these materials.

Coatings such as polymeric matrix materials and hydrogels can be applied in any suitable fashion. Known methods are dipping, coating, spraying, or impregnating the coating onto the harness. These coatings may be applied to the entire harness or selectively to the silicone jacketing, grip pads, or electrode locations. The coatings can be elastic and capable of handling the cyclic loading conditions on the heart. The coatings can be designed such that they do not affect the elastic, chronic fatigue, and performance characteristics of the harness.

Other potential coating methods include the deposition of a tether molecule, such as a peptide, to provide a site on the surface of the harness for attachment of the selected therapeutic medicament. Nano materials, virus or bacteria vectors, hydrogels, stem cells, hydroxypropylchitosan acetate, collagen, biostable and/or biodegradable polymers may also be used as carriers for drug delivery. Such carriers may sense epicardial gene expression or chemistry and tailor therapy accordingly.

Another possible method to facilitate surface delivery of selected medicaments is to provide a structure on the surface of the harness into which appropriately sized spaces are provided for the deposition and subsequent release of medicaments. Such release can be initiated by chemical reactions with elements on the myocardical surface, by dissolution of the medicaments in fluids that occur naturally inside the pericardium, or by pressure introduced through the structure of the harness externally, among others. Since the cardiac harness can be designed and delivered to provide a target therapeutic pressure “dose,” the harness can also be used for pressure mediated drug elution. Biodegradable or non-biodegradable hydrogels swell such that an aqueous therapeutic agent solution can be effectively squeezed out of the coating when pressure is applied, especially when the pericardium is left intact and covers the harness, thereby applying a slight compressive force on the harness, and hence the coating.

The beneficial medicaments applied through the methods listed above or other delivery means may be coated with a biodegradable material and/or surface that is designed to deteriorate at a pre-determined rate, such that the medicament is released to the surface of the heart, from the harness, over a pre-defined time period.

Possible biodegradable coatings are polylactides, polyglycolides, polycaprolactones, polyanhydrides, polyamides, polyurethanes, parylene, polyesteramides, polyorthoesters, polydioxanones, polyacetals, polyketals, polycarbonates, polyorthocarbonates, polyphosphazens, polyhydroxybutyrates, polyhdyroxyvalerates, polyalkylene oxalates, polyalkylene succinates, poly(malic acid), poly(amino acids), polyvinylpyrrolidone, polyethylene glycol, polyhydroxycellulose, chitin, chitosan, and copolymers, terpolymers, or combinations or mixtures of the above materials.

The mechanical energy of the heart during each cardiac cycle may also drive delivery. The micro-protruding self-anchoring features of the cardiac harness may be used for medicament delivery. These would effectively act as “microneedles” and inject the agents below the epicardial surface. Thus, as the heart beats during the cardiac cycle, the grip pads 27 (FIG. 5A) can have protrusions on the side facing the epicardium to help secure the harness on the heart and to act as “microneedles” to assist in delivery of a drug into the epicardium.

In yet another embodiment, the material coating 37 such as a dielectric coating in the cardiac harness 20 is itself impregnated with aldosterone blockade. In this embodiment, over time the aldosterone blockade elutes directly from the impregnated dielectric material.

In another embodiment, as shown in FIGS. 20-23, the electrodes 120 on the cardiac harness can have multiple lumens 125 for use in delivering a therapeutic amount of an mTOR inhibitor or aldosterone blockade directly onto the epicardial surface of the heart 10. Preferably, a therapeutic amount of aldosterone blockade is 0.1 to 200 mg. per day. An infusion pump or similar known device can be connected to lumens 125 to pump the mTOR inhibitor or aldosterone blockade through the lumens 125 and onto the epicardium. Again, the intact pericardium acts as a barrier to keep the drug in contact with the epicardium. Alternatively, the therapeutic agent may also be held in reservoirs or in others materials separate from the basic cardiac harness structure that could be delivered to the surface of the harness by flow channels constructed in the harness surface or through the lumbar 125. These reservoirs could be patches or bladders containing the therapeutic agent, preferably refillable. Refilling the bladder can be achieved in many ways (e.g. via a catheter connection or needle injection). The patch or bladder can be attached underneath the harness, within the harness, or at remote location. Acute beneficial medicament delivery can also be achieved during implant delivery from a reservoir contained on the delivery system for the cardiac harness.

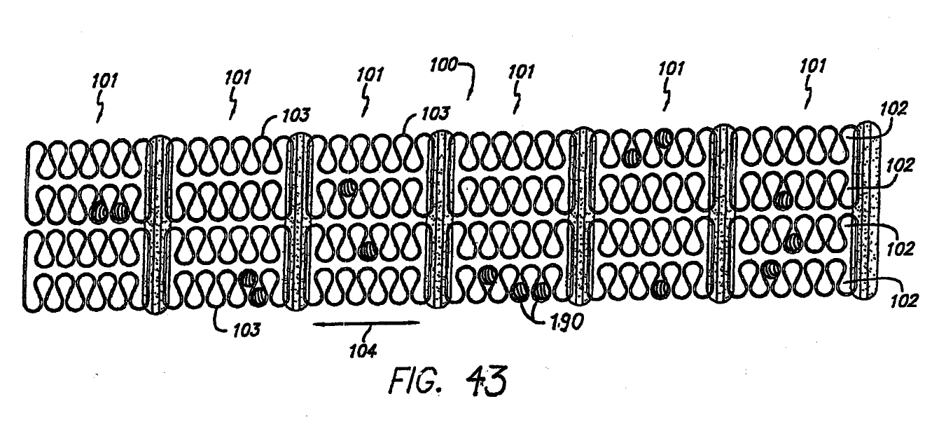

In yet another embodiment, as shown in FIG. 43, a free-standing biodegradable plug or implant 190 is attached to the undulations 103 of the cardiac harness 100. In one embodiment, the biodegradable plug or implant 190 consists of a beneficial medicament, such as an mTOR inhibitor or aldosterone blockade, whereby over time the medicament elutes, degrades, and separates itself from the plug or implant 190. In this embodiment, the plugs or implants 190 may be attached to the undulations 103 of the cardiac harness 100 in any order and in any number in a manner that achieves uniform elution of the medicament over the epicardium of the heart.

Alternatively, the implant 190 can contain cells for providing a therapeutic response. Cell populations can be attached to the harness by various means. Cells can be cultured directly onto the harness or developed into implant 190 and then attached to the undulations 103. The silicone rubber on the harness would act as a scaffold. The implanted cells may also serve as a platform for protein delivery at the surface of the heart (myocardial repair and enhance growth of the transplanted cells). They may be delivered via injection, via grip pads, or via exposure to the epicardial surface. Neurotrophic factors and/or angiogenic factors, such as vascular endothelial growth factor or fibroblast growth factor, can be locally expressed from these cells to avoid the potentially harmful effects of systemic delivery of these proteins.

The beneficial medicament may be one or more therapeutic genes. As used herein, the term “therapeutic gene” is a segment of nucleic acid that specifies a particular protein or polypeptide chain that, when expressed, provides a therapeutic effect. Many such therapeutic genes are known to prevent restenosis, promote angiogenesis, modulate pathways of electrical conductance to control cardiac arrhythmias, enhance the wound healing process, and/or express thrombolytic agents such as tissue plasminogen activator (TPA) or urokinase. They may be oligonucleotides, naked gene plasmids, ribozymes, or viral vectors containing specific genes. Possible delivery systems for these genes include: nanospheres, liposomes, microspheres, polymer matrices (biodegradable or non-biodegradable or a blend of the two), and naked nucleic acids. Therapeutic agents can be surface-acting or can penetrate the myocardium, coronary vessels, or surrounding tissue (e.g. small molecule compounds). When carriers are required to deliver the therapeutic agent, they may be designed to further reduce any undesirable side effects of the agent.

The beneficial medicament may also be one or more agents of cellular material. Cellular material can improve the function and structure of diseased tissue. The cellular material may be delivered via injection, via grip pads, or via exposure to the epicardial surface. Potential candidates for cellular material are: differentiated cells with different phenotypes (such as smooth muscle cells, endothelial cells, and fibroblasts); differentiated cells with the same phenotype (such as myocardial cells); non-differentiated cells, such as mesenchymal and other stem cells; cells that are xenogenic, allogenic and/or isogenic to the host; and genetically engineered cells.

Cellular material may be of a single tissue type or may contain a mixed population of cells. If desired, the culture media for the cells may also be delivered. This media may be supplemented as necessary with hormone and/or other growth factors, salts, buffers, nuclosides, antibiotics and trace elements (inorganic compounds usually present at final concentrations in the micromolar range).

Transdifferentiation may also be used as part of the therapy. Transdifferentiation involves the conversion of a committed, differentiated, or specialized cell to another differentiated cell type with a distinctly different phenotype. For myocytes, the cells can be made to contract synchronously.

Localized, targeted delivery of the beneficial medicament can avoid undesirable systemic effects by eliminating circulation of the drug in areas of the body other than the target tissue. Many existing heart failure medicaments have an improved effect on the heart at higher doses, but these doses are unusable due to the severity of the side effects (e.g. aldosterone antagonists and hyperkalemia). Lower amounts but potentially higher localized concentrations of the beneficial medicament can thus potentially be delivered without significant side effects.

Therapeutic agents can be added to the cardiac harness in a number of ways. The delivery dose can be based on time, a tethering molecule, or it may be based on “smart” signaling or sensing from the immediate environment or other systemic indicators. The release of an agent may be zero order, multi-phasic, or delayed. There may be an initial bolus dose of the therapeutic agent, followed by a relatively constant release of the agent over time.

Beneficial medicament delivery can be achieved through passive or active methods. Passive methods include diffusion from the delivery source. Active delivery mechanisms use an energy source to deliver the agent to the target tissue. Energy sources may be pumps or electrical current or osmosis. Other suitable external energy sources include ultrasound, thermal energy, radiofrequency, or microwave energy. The movement of the heart through each cardiac cycle can be used as an energy source (for bladder or coating or hydrogel delivery).

Durect Corporation has a number of potential reservoir-type delivery systems: the Duros osmotic pump, the SABER Depot Injection Technology, and the Durin Biodegradable Implants. The Durin product family allows high drug loading (up to 80%), is fully biodegradable (by hydrolysis), has a history of safe human use (lactide-glycolide co-polymers), will work with peptides, and allows for first order, zero order, delayed or biphasic drug release up to 6 months or more. The material used has been approved in over 30 medical devices and drug delivery systems.

All of the aforementioned methods would provide predictable release and delivery of beneficial medicament at the time of harness implantation and afterwards at an appropriate targeted dose and duration.

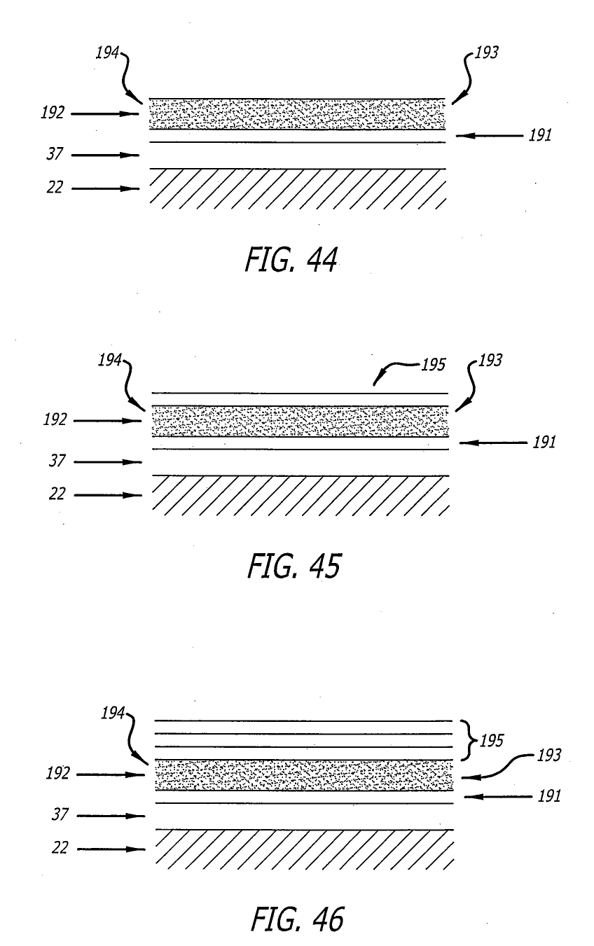

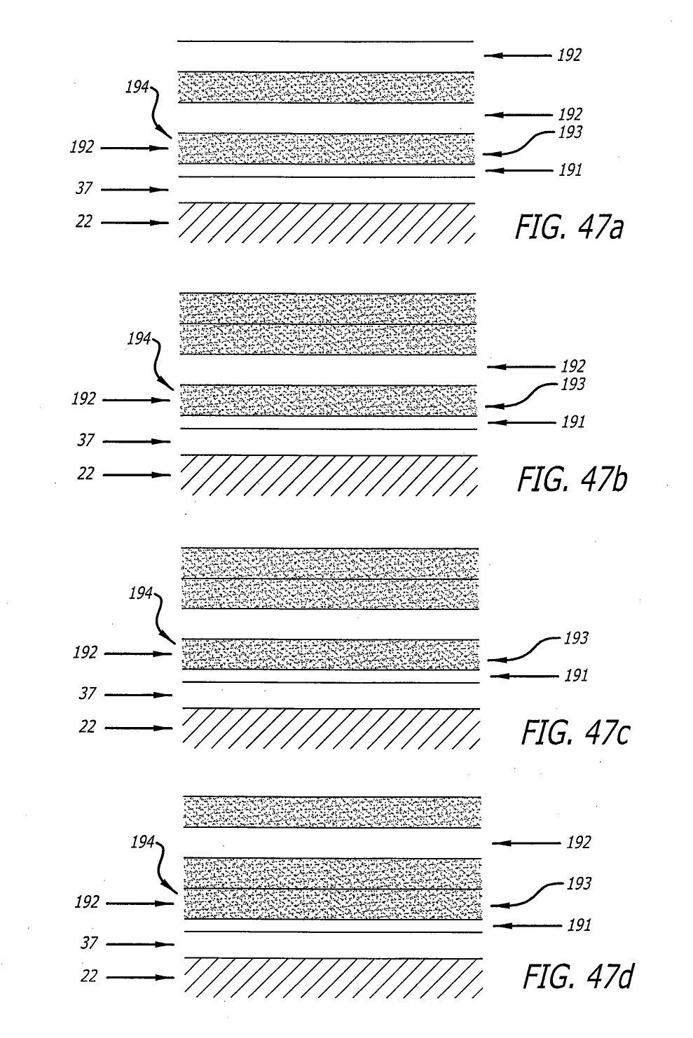

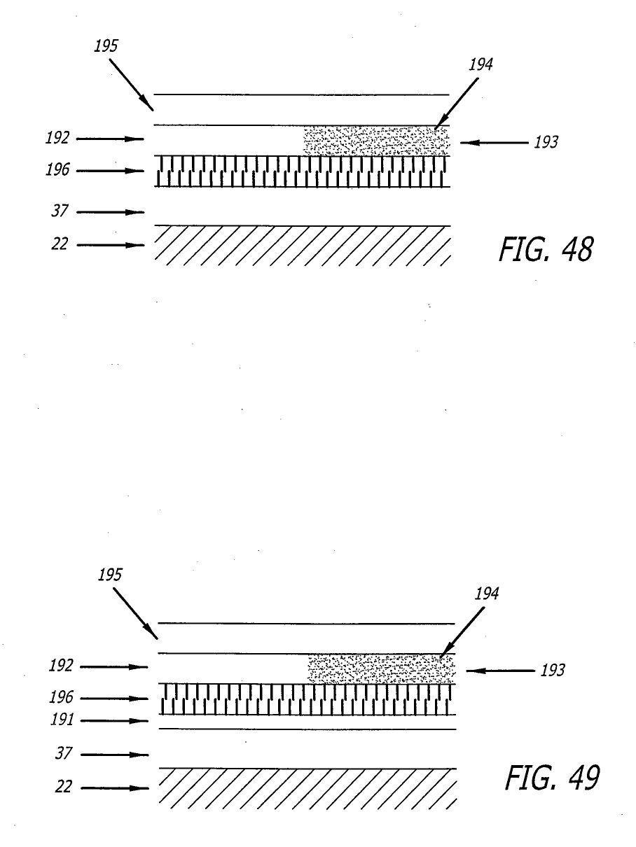

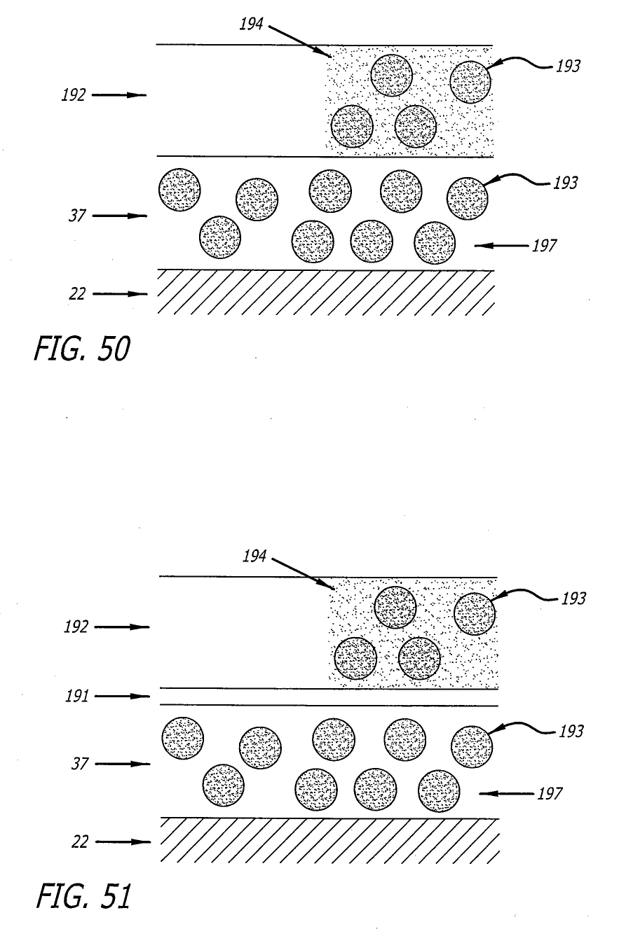

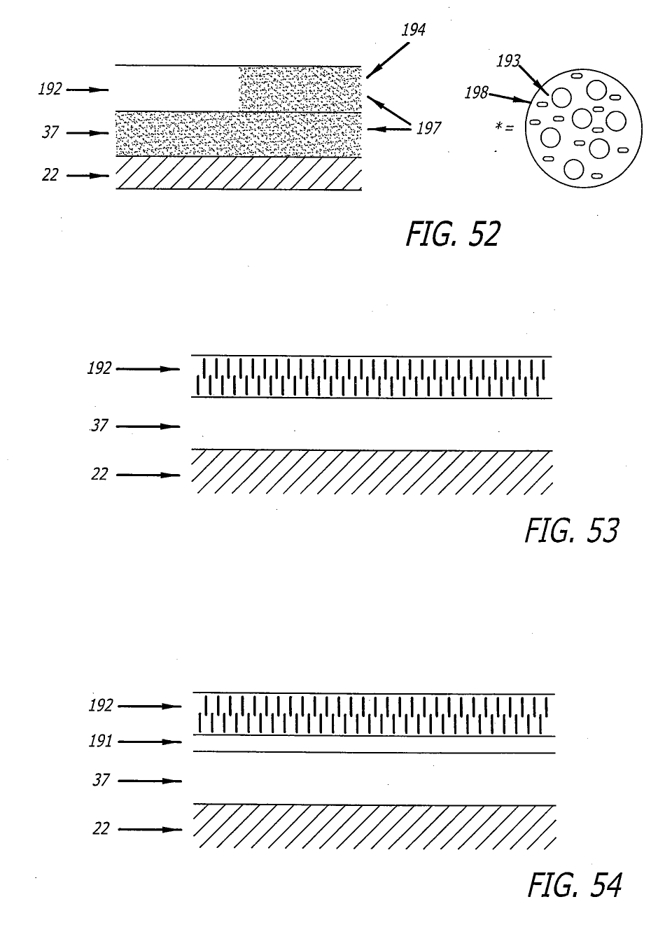

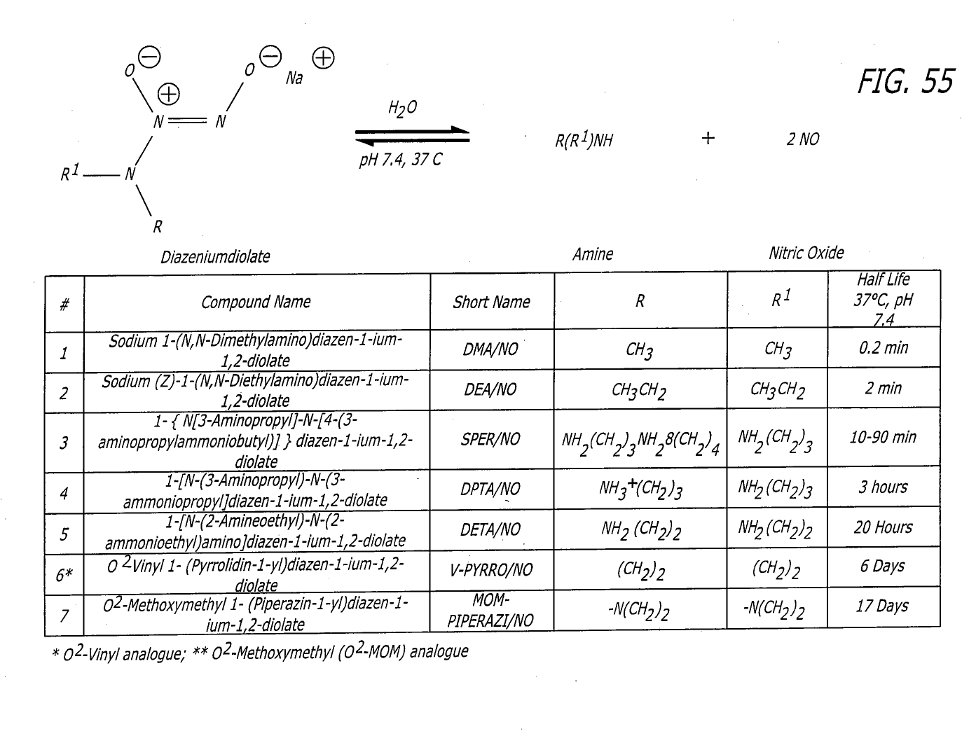

FIG. 44 illustrates one embodiment of the present invention wherein a cardiac harness having undulating strands 22 includes a dielectric layer 37 formed thereon. The dielectric layer 37 thereupon has a tie layer 191 which binds the a layer of therapeutic agent 193 and polymer matrix 192. An optional plasticizer 194 may also be incorporated over the therapeutic agent 193 and polymer matrix 192. In FIG. 45, the embodiment of FIG. 44 has a polymer top coat 195 added over the plasticizer 194. FIG. 46 shows the same embodiment with multiple top coats 195. In FIG. 47, the harness is shown with multiple or alternating layers of therapeutic agent 193 and polymer matrix 192, successively or separated by other layers. In FIG. 48, a layer of second therapeutic agent 196 is disposed between the dielectric layer and the first therapeutic agent 193, and FIG. 49 shows the same embodiment with a tie layer 191 interposed between the dielectric layer 37 and the second therapeutic agent 196. FIG. 50 illustrates an embodiment where microspheres 197 are disposed in the dielectric layer 37 and the polymer matrix/therapeutic agent layers with a plasticizer also included in the upper layer. FIG. 51 includes a tie layer 191 to the embodiment of FIG. 50 between the dielectric layer 37 and the polymer matrix 192 with therapeutic agent 193 layers. FIG. 52 includes a dielectric layer 37 above the undulating strands 22, where the dielectric layer includes microspheres including therapeutic agent 193 and nanoparticles 198, and the polymer matrix 192 also includes the microspheres along with an optional plasticizer. FIG. 53 and FIG. 54 depict layers of a dielectric 37 and a therapeutic agent 193, without and with a tie layer 191 respectively.

It may be desired to reduce the likelihood of the development of fibrotic tissue over the cardiac harness so that the elastic properties of the harness are not compromised. As fibrotic tissue increases, the right and left ventricular thresholds may increase, commonly referred to as “exit block.” When exit block is detected, the pacing therapy may have to be adjusted. Certain drugs such as steriods, have been found to inhibit cell growth leading to scar tissue or fibrotic tissue growth. Examples of therapeutic drugs or pharmacologic compounds that may be loaded onto the cardiac harness or into a polymeric coating on the harness, on a polymeric sleeve, on individual undulating strands on the harness, or infused through the lumens in the electrodes and delivered to the epicardial surface of the heart include steroids, taxol, aspirin, prostaglandins, and the like. Various therapeutic agents such as antithrombogenic or antiproliferative drugs are used to further control scar tissue formation. Examples of therapeutic agents or drugs that are suitable for use in accordance with the present invention include 17-beta estradiol, sirolimus, everolimus, actinomycin D (ActD), taxol, paclitaxel, or derivatives and analogs thereof. Examples of agents include other antiproliferative substances as well as antineoplastic, anti-inflammatory, antiplatelet, anticoagulant, antifibrin, antithrombin, antimitotic, antibiotic, and antioxidant substances. Examples of antineoplastics include taxol (paclitaxel and docetaxel). Further examples of therapeutic drugs or agents include antiplatelets, anticoagulants, antifibrins, anti-inflammatories, antithrombins, and antiproliferatives. Examples of antiplatelets, anticoagulants, antifibrins, and antithrombins include, but are not limited to, sodium heparin, low molecular weight heparin, hirudin, argatroban, forskolin, vapiprost, prostacyclin and prostacyclin analogs, dextran, D-phe-pro-arg-chloromethylketone (synthetic antithrombin), dipyridamole, glycoprotein IIb/IIIa platelet membrane receptor antagonist, recombinant hirudin, thrombin inhibitor (available from Biogen located in Cambridge, Mass.), and 7E-3B® (an antiplatelet drug from Centocor located in Malvern, Pa.). Examples of antimitotic agents include methotrexate, azathioprine, vincristine, vinblastine, fluorouracil, adriamycin, and mutamycin. Examples of cytostatic or antiproliferative agents include angiopeptin (a somatostatin analog from Ibsen located in the United Kingdom), angiotensin converting enzyme inhibitors such as Captopril® (available from Squibb located in New York, N.Y.), Cilazapril® (available from Hoffman-LaRoche located in Basel, Switzerland), or Lisinopril® (available from Merck located in Whitehouse Station, N.J.); calcium channel blockers (such as Nifedipine), colchicine, fibroblast growth factor (FGF) antagonists, fish oil (omega 3-fatty acid), histamine antagonists, Lovastatin® (an inhibitor of HMG-CoA reductase, a cholesterol lowering drug from Merck), methotrexate, monoclonal antibodies (such as PDGF receptors), nitroprusside, phosphodiesterase inhibitors, prostaglandin inhibitor (available from GlaxoSmithKline located in United Kingdom), Seramin (a PDGF antagonist), serotonin blockers, steroids, thioprotease inhibitors, triazolopyrimidine (a PDGF antagonist), and nitric oxide. Other therapeutic drugs or agents which may be appropriate include alpha-interferon, genetically engineered epithelial cells, and dexamethasone.

Diazeniumdiolates, more commonly referred to as NONOates, have been extremely useful in the investigation of the biological effects of nitric oxide (NO) and related nitrogen oxides. The NONOate releases nitric oxide under physiological conditions and exhibits unique cardiovascular features that may have relevance for pharmacological treatment of heart failure. FIG. 55 illustrates an exemplary chemical reaction. Table I below illustrates various compounds investigated.

TABLE I

| | | | | | Half Life | |
|---|---|---|---|---|---|---|
| | | | | | 37° C., | |
| # | Compound Name | Short Name | R | R¹ | pH 7.4 | |
| 1 | Sodium 1-(N,N- | DMA/NO | CH₃ | CH₃ | 0.2 | min |
| | Dimthylanimo)diazen-1-ium-1,2- | | | | | |
| | dilate | | | | | |
| 2 | Sodium (Z)-1-(N,N- | DEA/NO | CH₃CH₂ | CH₃CH₂ | 2 | min |
| | Dimthylanimo)diazen-1-ium-1,2- | | | | | |
| | dilate | | | | | |
| 3 | 1-{N-[3-Aminopropyl]-N-[4-(3- | SPER/NO | NH₂(CH₂)₃ | NH₂(CH₂)₃ | 10-90 | min |
| | aminopropylammoniobutyl)]}diazen- | | NH₂⁺ (CH₂)₄ | | | |
| | 1-ium-1,2-diolate | | | | | |
| 4 | 1-[N-(3-Aminopropyl)-N-(3- | DPTA/NO | NH₃⁺ (CH₂)₃ | NH₂(CH₂)₃ | 3 | hrs |
| | ammoniopropyl]diazen-1-ium-1,2- | | | | | |
| | diolate | | | | | |
| 5 | 1-[N-(2-Aminoethyl)-N-(2- | DETA/NO | NH₂ (CH₂)₂ | NH₂(CH₂)₂ | 20 | hrs |
| | ammonioethyl)amino]diazen-1-ium- | | | | | |
| | 1,2-diolate | | | | | |
| 6* | O²-Vinyl 1-(Pyrrolidin-1-yl)diazen- | V-PYRRO/ | (CH₂)₂ | (CH₂)₂ | 6 | days |
| | 1-ium-1,2-dilate | NO | | | | |
| 7** | O²-Methozymethyl 1-(Piperazin-1- | MOM- | —N(CH₂)₂ | —N(CH₂)₂ | 17 | days |
| | yl)diazen-1-ium-1,2-diolate | PIPERAZI/NO | | | | |

*O²-Vinyl analogue;
**O²-Methoxymethyl (O²-MOM) analogue

The beneficial medicament may be delivered to one or more specific target areas on or around the heart, or the entire surface of the heart can be treated. An example of a potential specific target area is an ischemic zone with poor blood flow. A combination of therapeutic agents may be used independently or overlapping in target areas (e.g. simultaneous use of aldosterone antagonists and anti-arrhythmic drugs). After delivery to the target tissue, the therapeutic agent can penetrate the tissue surface and act below the surface of the tissue. The beneficial medicaments may be released only in the direction of the heart or they may be released more universally within the pericardial space.

Although the present invention has been described in terms of certain preferred embodiments, other embodiments that are apparent to those of ordinary skill in the art are also within the scope of the invention. Further, none of the above disclosures or embodiments should be limited to treatment of the heart. The device and method can be used to treat tissues surrounding the heart or other tissues of the body, as desired. Accordingly, the scope of the invention is intended to be defined only by reference to the appended claims. While the dimensions, types of materials and coatings described herein are intended to define the parameters of the invention, they are by no means limiting and are exemplary embodiments.

What is claimed:

1. A cardiac harness for drug delivery, comprising:
a cardiac harness having a coating of a polymer material;
an mTOR inhibitor impregnated into the polymer material; and
the mTOR inhibitor configured to elute from the polymer material in a predetermined dose range during a time period of one day to two years.

2. The cardiac harness of claim 1, wherein the polymer material includes poly-butyl methacrylate and polyethylene vinyl acetate.

3. The cardiac harness of claim 1, wherein the polymer material is coated onto the cardiac harness and the mTOR inhibitor is impregnated into the polymer material.

4. A drug coated cardiac harness, comprising:
a cardiac harness having a coating of a dielectric material;
a coating of blockade on the dielectric material; and
the coating of the blockade configured to elute in a dose range of 0.1 mg to 200 mg per day.

5. The drug coated cardiac harness of claim 4, wherein the dielectric material consists of silicone rubber.

6. The drug coated cardiac harness of claim 4, wherein the dielectric material is impregnated with the blockade material.

7. A method of delivering a drug using a cardiac harness, comprising:
coating blockade on the surface of a cardiac harness for delivering the drug to the epicardial surface in a dose range between 0.1 mg to 200 mg per day.

8. The method of claim 7, wherein the blockade coating is non-biodegradable.

9. The method of claim 7, wherein the blockade coating is biodegradable.

10. The method of claim 7, wherein the blockade is impregnated into a dielectric material on the cardiac harness.

11. The method of claim 7, wherein the blockade is formed into an implant attached to the cardiac harness.

12. A drug coated cardiac harness, comprising:
a cardiac harness comprising undulating strands having a coating of a dielectric material;
a layer comprising a polymer matrix and a therapeutic agent disposed over the dielectric layer.

13. The drug coated cardiac harness of claim 12 further comprising a polymer top coat layer.

14. The drug coated cardiac harness of claim 12 further comprising a plasticizer.

15. The drug coated cardiac harness of claim 12 further comprising alternate layers of plasticizer and therapeutic agent with polymer matrix.

16. The drug coated cardiac harness of claim 12 further comprising a layer of a second therapeutic agent.

17. The drug coated cardiac harness of claim 16 further comprising a tie layer.

18. The drug coated cardiac harness of claim 12 wherein the dielectric layer includes microspheres having a therapeutic agent thereon.

19. The drug coated cardiac harness of claim 18 wherein the microspheres also are disposed in a polymer matrix layer.

20. The drug coated cardiac harness of claim 19 further including a tie layer between the dielectric layer and the polymer matrix layer.

21. The drug coated cardiac harness of claim 19 wherein the microspheres include nanoparticles dispersed between the therapeutic agent.
